(19) United States
(12) Patent Application Publication
(10) Pub. No.: US 2010/0029562 A1
(43) Pub. Date: Feb. 4, 2010

(54) hG31P Expression System

(76) Inventors: John R. Gordon, Saskatoon (CA); David J. Cheng, Coquitlam (CA)

Correspondence Address: ADE & COMPANY INC., 2157 Henderson Highway, WINNIPEG, MB, R2G1P9, CA

(21) Appl. No.: 12/470,275

(22) Filed: May 21, 2009

Related U.S. Application Data
(60) Provisional application No. 61/055,043, filed on May 21, 2008.

(51) Int. Cl. A61K 38/16; C12N 15/63; C12P 21/00; A61P 17/00; A61P 11/00; A61P 1/00; A61P 9/00; A61P 25/00; A61P 25/28; A61P 19/02

(52) U.S. Cl. 514/12; 435/320.1; 435/69.1

(57) ABSTRACT

Expression plasmids and expression systems for the expression of human G31P⁺² are described.

### PRIOR APPLICATION INFORMATION

The instant application claims the benefit of U.S. Provisional Patent Application 61/055,043, filed May 21, 2008.

### BACKGROUND OF THE INVENTION

CXC chemokines that posses the ELR motif are important to the influx of inflammatory cells. In many diseases, the pathology is in fact the result of overexpression or prolonged expression of inflammatory cells and accordingly the discovery of therapeutic agents capable of blocking ELR chemokines has become a research priority.

For example, it is known that when amino terminal truncation of bovine CXCL8 is combined with a lysine to arginine substitution at amino acid 11 (i.e., CXCL8(3-74)K11R), dramatic increases in CXCR1 and CXCR2 receptor affinity are evident, such that CXCL8(3-74)K11R competitively inhibits the binding of multiple ligands to both receptors (Li, F., and J. R. Gordon. 2001. Biochem. Biophys. Res. Comm. 286:595-600., hereby incorporated by reference).

However, bovine-based peptides are undesirable therapeutics for humans.

The CXC chemokines that possess the receptor-signaling glutamic acid-leucine-arginine (ELR) motif (e.g., CXCL1/GROα, CXCL8/IL-8; Baggiolini, M. 1998. Nature. 392:565-568) are important to the influx of inflammatory cells that mediates much of the pathology in multiple settings, including ischemia-reperfusion injury (Sekido, N. et al. 1993. Nature. 365:654-657; Villard, J. et al. 1995. Am. J. Respir. Crit. Care Med. 152:1549-1554), endotoxemia-induced acute respiratory distress syndrome (ARDS; Mukaida, N. et al. 1998. Inflamm. Res. 47 suppl. 3):S151-157), arthritis, and immune complex-type glomerulonephritis (Harada, A. et al. 1996. Inflamm. Res. 2:482-489). For instance, inappropriately released hydrolytic enzymes and reactive oxygen species from activated neutrophils initiate and/or perpetuate the pathologic processes. On the other hand, during most bacterial infections this chemokine response represents a critical first line of defense. But even here, ELR⁺ CXC chemokine responses can, via their abilities to activate inflammatory cells displaying the CXCR1 and CXCR2 receptors, exacerbate the pathology. For example, during experimental ‘cecal puncture and ligation’ sepsis, neutralization of MIP-2 reduces mouse mortality from 85 to 38% (Walley, K. R. et al. 1997. Infect. Immun. 65:3847-3851). And experimental treatments that eliminate circulating neutrophils ameliorate the pathology of pneumonic mannheimiosis (Slocombe, R. et al. 1985. Am. J. Vet. Res. 46:2253), wherein CXCL8 expression in the airways variably affects the neutrophil chemoattraction. (Caswell, J. L. et al. 1997. Vet. Pathol. 35:124-131; Caswell, J. L. et al. 2001. Canad. J. Vet. Res. 65:229-232). Despite the critical importance of these chemokine responses in many settings, wayward inflammatory cell responses are sufficiently damaging that the development of therapeutic tools with which we can block ELR⁺ chemokines has become a research priority (Baggiolini, M., and B. Moser. 1997. J. Exp. Med. 186:1189-1191).

The ‘ELR’ chemokines chemoattract and activate inflammatory cells via their CXCR1 and CXCR2 receptors (Baggiolini, 1998; Ahuja, S. K., and P. M. Murphy. 1996. J. Biol. Chem. 271:20545-20550). Most mammals express orthologs (genes in different species that evolved from a common ancestral gene by speciation) of the CXCR1 and CXCR2 receptors and the ‘ELR’ chemokines. Sequence similarity between these homologous (genetically or functionally related) genes is high; higher still when conserved amino acid substitutions are considered. Mouse and rat are exceptions where these species do not have CXCR1 genes and their CXCL8 equivalent is highly divergent from that of other mammals. Interleukin 8 (CXCL8) is not species specific, in that the CXCL8 protein from one species can be functional in another species (Rot, 1991, Cytokine 3: 21-27).

The CXCR1 is specific for CXCL8 and CXCL6/granulocyte chemotactic protein-2 (GCP-2), while the CXCR2 binds CXCL8 with high affinity, but also macrophage inflammatory protein-2 (MIP-2), CXCL1, CXCL5/ENA-78, and CXCL6 with somewhat lower affinities (see, for example, Baggiolini and Moser, 1997). CXCL8 signaling in cell lines transfected with the human CXCR1 or CXCR2 induces equipotent chemotactic responses (Wuyts, A. et al. 1998. Eur. J. Biochem. 255:67-73; Richardson, R. et al. 1998. J. Biol. Chem. 273:23830-23836), and while neutrophil cytosolic free Ca⁺⁺ changes and cellular degranulation in response to CXCL8 are also mediated by both receptors, the respiratory burst and activation of phospholipase D reportedly depend exclusively on the CXCR1 (Jones, S. A. et al. 1996. Proc. Natl. Acad. Sci. U.S.A. 93:6682-6686.). On the other hand, it has been reported that a non-peptide antagonist of the CXCR2, but not the CXCR1, antagonizes CXCL8-mediated neutrophil chemotaxis, but not cellular activation (White, J. R. et al. 1998. J. Biol. Chem. 273:10095-10098.). Finally, there is abundant evidence that chemokines are most often redundantly expressed during inflammatory responses (see, for example, Caswell et al., 1997). But, despite active research in the field, no CXC chemokine antagonists are known in the prior art that are effective in suppressing adverse inflammatory cell activity induced by either ELR-CXC chemokine receptor.

### SUMMARY OF THE INVENTION

According to a first aspect of the invention, there is provided an expression vector comprising a polynucleotide sequence deduced from an amino acid sequence as set forth in SEQ ID No. 1 operatively linked to a suitable promoter.

According to a second aspect of the invention, there is provided a method of producing hG31P⁺² peptide comprising:

transforming a suitable host cell with an expression vector comprising a polynucleotide sequence deduced from an amino acid sequence as set forth in SEQ ID No. 1 operatively linked to a suitable promoter functional in said host;

growing the host cell under conditions promoting expression of the hG31P⁺²; and

recovering the hG31P⁺² from the host cell.

According to a third aspect of the invention, there is provided a method of treating a CXC chemokine-mediated disease comprising administering to an individual in need of such treatment an effective amount of a peptide produced according to the method described above.

### BRIEF DESCRIPTION OF THE DRAWINGS

FIG. 1. The G31P analogue of CXCL8₍₃₋₇₄₎K11R is a potent inhibitor of CXCL8-binding to peripheral blood neutrophils. Bovine peripheral blood neutrophils (87-93% purity) were (upper panel) exposed at 4° C. for 2 h to CXCL8₍₃₋₇₄₎K11R analogues (10 ng/ml) or medium (med) alone, then washed and similarly incubated with biotinylated CXCL8 (^{biot}CXCL8; 1000 ng/ml or 129 nM). These levels of CXCL8 approximate those found in the lung tissues of animals with pneumonic pasteurellosis (ref. 8, 9). The levels of ^{biot}CXCL8 binding to the cells were determined using ELISA technology. The depicted amino acid substitutions within CXCL8₍₃₋₇₄₎K11R included: G31P; P32G; T12S/H13P/G31P; and T12S/H13P/G31P/P32G. The G31P, but not the P32G, analogue was a highly effective antagonist of CXCL8 binding to the cells. With both the G31P and P32G analogues, additional substitutions of T12S and H13F reduced their CXCL8 antagonist activities (lower panel). Neutrophils were exposed simultaneously for 45 min at 4.degree. C. to varying concentrations of CXCL8₍₃₋₇₄₎K11R/G31P or unlabeled CXCL8 and 20 pM ¹²⁵ICXCL8. This level of ¹²⁵I-CXCL8 was chosen as nearly saturating for the cell's high affinity CXCL8 receptors (data not shown). The levels of cell-associated ¹²⁵I-CXCL8 were assessed using a counter. The data clearly indicate that CXCL8₍₃₋₇₄₎K11R/G31P had a substantially higher affinity for the neutrophils than CXCL8.

FIG. 2. CXCL8₍₃₋₇₄₎K11R/G31P is not an agonist of neutrophil chemoattraction responses or -glucuronidase release. CXCL8 and the G31P, P32G, or combined G31P/P32G analogues of CXCL8₍₃₋₇₄₎K11R were tested for their neutrophil agonist activities, using freshly purified bovine peripheral blood neutrophils. (upper panel) The chemotactic responses to each protein were tested in 30 min microchemotaxis assays and the results expressed as the mean (+/−SEM) number of cells/40× objective microscope field, as outlined in the methods section. Both the G31P and G31P/P32G analogues displayed little discernable chemotactic activity, while the P32G analogue stimulated substantial responses at 100 ng/ml. (lower panel) The neutrophils were exposed to varying doses of each analogue for 30 min, then the cellular secretion products were assayed for -glucuronidase using the chromogenic substrate p-nitrophenyl-D-glucuronide, as presented in the methods section. The total cellular stores of -glucuronidase were determined from aliquots of cells lysed with Triton-X-100. The enzyme release with each treatment is expressed as the percent of the total cellular stores. None of the analogues had substantial agonist activity, although CXCL8 itself did induce significant enzyme release. The positive control treatment with phorbol-12,13-myristate acetate and calcium ionophore A23187 induced 42+/−6% enzyme release.

FIG. 3 CXCL8₍₃₋₇₄₎K11R-G31P is a highly effective antagonist ELR-CXC chemokine-medicated neutrophil chemoattraction. The ability of CXCL8₍₃₋₇₄₎K11R/G31P to block chemotactic responses of bovine neutrophils to several ELR-CXC chemokines was measured using 20 min microchemotaxis assays. (left panel) The cells were simultaneously exposed to CXCL8 (1 μg/ml) and varying concentrations of the analogue. The number of cells that responded to the CXCL8 was assessed by direct counting of the chemotaxis assay membranes, as in FIG. 2. CXCL8₍₃₋₇₄₎K11R/G31P was a highly effective competitive inhibitor of the cell's responses to CXCL8. (middle panel) Dose-response curves for chemoattraction of bovine neutrophils by human CXCL1, CXCL5, or CXCL8. Each chemokine displayed a biphasic activity pattern, with maxima at 1-10 ng/ml and at 1 μg/ml. (right panel) The ability of CXCL8₍₃₋₇₄₎K11R/G31P to block the cell's responses to 1 ng/ml of human CXCL5 or CXCL1 or 10 ng/ml of human CXCL8 was assessed as above. CXCL8₍₃₋₇₄₎K11R/G31P effectively antagonized each ELR-CXC chemokine, with complete inhibition being achieved with from 3-20 nM CXCL8₍₃₋₇₄₎K11R/G31P.

FIG. 4. CXCL8₍₃₋₇₄₎K11R-G31P blocks the activities of CXCL8 and non-CXCL8 chemoattractants expressed within pneumonic airways or in endotoxin-induced mastitis. The effects of monoclonal anti-IL8 antibody 8B6 or CXCL8₍₃₋₇₄₎K11R-G31P on neutrophil responses to the chemoattractants expressed within the airways of animals with pneumonic pasteurellosis or in the mammary cisterns of cattle with endotoxin-induced mastitis were assessed as in FIG. 3. (A) Diluted (1:10) bronchoalveolar lavage fluids (BALF) from lesional lung lobes of pneumonic cattle (PNEUMONIA) or teat cistern lavage fluids from cattle with mastitis (MASTITIS) were tested as is (none) or after treatment with either anti-CXCL8 MAb 8B6 (5 μg/ml) or CXCL8₍₃₋₇₄₎K11R/G31P (G31P; 1 or 10 ng/ml) for their chemotactic activities compared to medium alone. With both samples, the Mab 8B6 antibodies by themselves neutralized 74% of the chemotactic activities in the samples, while CXCL8₍₃₋₇₄₎K11R/G31P reduced the responses by 93-97%. (B) In order to confirm these results using an alternate strategy, we next absorbed lesional BAL fluids with monoclonal antibody 8B6-immunoaffinity matrices, removing >99% of their content of CXCL8, then tested both their residual chemotactic activities and the ability of CXCL8₍₃₋₇₄₎K11R/G31P to antagonize these residual non-CXCL8 chemotactic activities. There was a dose-dependent inhibition of the total and residual chemotactic activities in the samples, indicating that both CXCL8 and non-CXCL8 chemoattractants are expressed in these lesions.

FIG. 5. CXCL8₍₃₋₇₄₎K11R-G31P can ablate endotoxin-induced inflammatory responses in vivo. Two week-old Holstein calves were tested for their neutrophilic inflammatory responses to intradermal endotoxin (1 μg/site) challenge before and at various time after intravenous (i.v.), subcutaneous (subcutan.), or intramuscular (i.m.) injection of CXCL8₍₃₋₇₄₎K11R-G31P (75 μg/kg). Fifteen hour endotoxin reaction site biopsies were obtained at 0, 16, 48 and 72 h post-treatment and processed for histopathologic assessment of the neutrophil response, as determined by counting the numbers of neutrophils in nine 40× objective microscope fields per section. (left panel) Photomicrographs of the tissue responses to endotoxin challenge around blood vessels within the reticular dermis prior to (0 h) and 48 h post-treatment. Large numbers of neutrophils accumulated around the vasculature within the reticular dermis in the pre-, but not post-treatment tissues. (B) Graphic presentation of the neutrophil responses to endotoxin challenge either before (0 h) or after (16, 48, 72 h) CXCL8₍₃₋₇₄₎K11R-G31P delivery by each route. ** or ***=p 0.01 or 0.001, respectively, relative to the internal control pretreatment responses.

FIG. 6 Eosinophils purified from the blood of atopic asthmatic or atopic non-asthmatic donors (left panels) or a subject with a hypereosinophilia (right panel) were assessed for their responses to recombinant human CXCL8, CXCL5, or CCL11, in the presence or absence of the indicated doses of recombinant bovine CXCL8₍₃₋₇₄₎K11R/G31P (G31P). Low doses of G31P were able to block the responses of these cells to each of the CXCR1 and CXCR2 ligands, but had no effect on the eosinophil's responses to the unrelated CCR3 ligand CCL11/eotaxin.

FIG. 7 Neutrophils from the peripheral blood of a healthy donor were tested for their responses to recombinant human CXCL8 or CXCL5 in the presence or absence of bovine CXCL8₍₃₋₇₄₎K11R/G31P (G31P; 10 ng/ml). G31P blocked the neutrophil's responses to both ligands.

FIG. 8 Physical characterization of hbG31P isoforms.

FIG. 9 Comparison of bG31P and hbG31P (CXCL8 antagonism)

FIG. 10A hbG31P analogues are not neutrophil agonists. FIG. 10B hbG31P antagonist activity.

FIG. 11 Effect of the carboxy terminal sequence of hbG31P on its antagonist activities.

FIG. 12 Agonist and CXCL8 analogues—activities of constructs

FIG. 13 Comparison of the antagonist activities of bG31P and hG31P+6

FIG. 14 hG31P antagonizes ENA78, a CXCR2-specific ligand

FIG. 15 hG31P antagonizes CXCL8-induced ROI release

FIG. 16 hG31P antagonizes neutrophil chemotactic activities in sputum of bronchiectasis and cystic fibrosis patients.

FIG. 17 hG31P reduces pulmonary inflammation in endotoxemic mice.

FIG. 18 Effect of hG31P in morbid airway endotoxemia in guinea pigs.

FIG. 19 Effect of various hG31P isoforms (and hbG31P) on airway endotoxemia pathology in guinea pigs.

FIG. 20 Effect of various hG31P isoforms (and hbG31P) on airway endotoxemia pathology in guinea pigs.

FIG. 21 hbG31P mutants effect on blocking human neutrophil (PMN) intracellular Ca influx response to human IL-8 and GROa. The activity of hbG31P and its mutants' activity on blocking PMN intracellular Ca release [Ca]ᵢ stimulated by human IL-8 and GROα were assessed. 100 ng/ml of bG31P, hbG31P, and hbG31P mutants were incubated with 2 μM Fluo-4 μM stained with 2×10⁵ PMNs for 15 mins, then stimulated by 100 ng/ml IL-8 or GROα. Then read the fluorescence of samples with fluorometer. The data were expressed as the area of [Ca]ₗ measurements. Generally, hbG31P mutants as well as hbG31P, bG31P showed effective agonist activity on PMN [Ca]ₗ stimulated by IL-8 and GROα.

FIG. 22 hbG31P mutants showed different effects on blocking PMN migration to inflammation site in skin test stimulated by LPS. hbG31P and its mutants' antagonist activity on blocking PMN infiltration into LPS stimulated inflammation site were assessed. The data were expressed as the percentage of the blocked number of PMN in 40× field of microscope of subcutaneous and intradermal tissue treated by 0.5 μg LPS and 250 μg/ml bG31P, hbG31P or its mutants in the number of PMN in LPS only treated inflammation site. The data clearly showed that hbG31P has equal blocking activity with bG31P. H13Y showed much better antagonist activity than T3K, E35A. T15K showed slight effect on blocking. Meanwhile, S37T doesn't show any antagonist activity.

FIG. 23. DNA sequence of the pJ5:G03799 plasmid (SEQ ID No. 3).

FIG. 24. Schematic diagram of the pET-22bhG31P⁺² plasmid.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

Described herein is the generation of expression plasmids for the expression of human G31P⁺² The amino acid sequence of human G31P⁺² is:

### GSKELRCQCIRTYSKPFHPKFIKELRVIESPPHCANTEIIVKLSDGRELCLDPKENWV QRVVEKFLKRAENS (SEQ ID No. 1)

As will be appreciated by one of skill in the art, a polynucleotide deduced from the amino acid sequence as set forth in SEQ ID No. 1 may be operatively linked to a suitable promoter for expression in a suitable host. The transgenic peptide produced can then be recovered from the host.

In an alternative embodiment, the nucleotide sequence as set forth in SEQ ID No. 2 is operatively linked to a suitable promoter for expression in a suitable host. The transgenic peptide produced can then be recovered from the host.

hK11RG31P⁺²

(SEQ ID No. 2)

GGCTCTAAAGAACTGCGTTGTCAATGCATTCGTACTTACTCTAAGCCATT
CCACCCGAAGTTCATCAAAGAACTGCGTGTGATTGAATCTCCGCCACACT
GCGCCAATACCGAAATCATTGTTAAACTGAGGGACGGTCGTGAACTGTGT
CTGGACCCGAAAGAAAATTGGGTACAGCGTGTGGTGGAAAAATTTCTGAA
ACGTGCCGAAAACTCT

Examples of suitable expression systems and suitable hosts are well known in the art. However, not all expression systems and/or hosts are suitable for expression of all peptides. For example, the peptide may be toxic to the host or may be cleaved or otherwise modified by the host cells such that functional or useful peptides cannot be recovered. Yet further, while certain expression systems may produce sufficient levels of peptide at an experimental or trial volume for the process to appear to be commercially viable, it is often found that increasing the culture volumes for commercial-scale production significantly reduces the yield of purified peptide per culture volume unit, often to a point that the process is no longer commercially viable.

In the examples below, exemplary protocols are provided for the production and purification of hG31P⁺². As will be appreciated by one of skill in the art, these protocols are intended to be illustrative and are not necessarily limiting. Specifically, it is of note that some variation and modification may be made to the various steps that will not significantly modify the levels of peptide produced.

As discussed below, the protocols described herein can be used to produce purified peptide at a level of approximately 10-15 mg per liter of cell culture.

As will be appreciated by one of skill in the art, peptides purified as described herein can be used in the manufacture of pharmaceutical compositions. Such compositions may be used in the treatment of CXC chemokine-mediated pathologies, for example, ELR-CXC chemokine-mediated diseases or pathologies. The chemokine mediated disease is preferably selected from the group consisting of psoriasis, atopic dermatitis, osteo arthritis, rheumatoid arthritis, asthma, chronic obstructive pulmonary disease, adult respiratory distress syndrome, inflammatory bowel disease, Crohn's disease, ulcerative colitis, stroke, septic shock, multiple sclerosis, endotoxic shock, gram negative sepsis, toxic shock syndrome, cardiac and renal reperfusion injury, glomerulonephritis, thrombosis, graft vs. host reaction, Alzheimer's disease, allograft rejections, malaria, restenosis, angiogenesis, atherosclerosis, osteoporosis, gingivitis and undesired hematopoietic stem cells release and diseases caused by respiratory viruses, herpes viruses, and hepatitis viruses, meningitis, cystic fibrosis, pre-term labor, cough, pruritus, multi-organ dysfunction, trauma, strains, sprains, contusions, psoriatic arthritis, herpes, encephalitis, CNS vasculitis, traumatic brain injury, CNS tumors, subarachnoid hemorrhage, post surgical trauma, interstitial pneumonitis, hypersensitivity, crystal induced arthritis, acute and chronic pancreatitis, acute alcoholic hepatitis, necrotizing enterocolitis, chronic sinusitis, uveitis, polymyositis, vasculitis, acne, gastric and duodenal ulcers, celiac disease, esophagitis, glossitis, airflow obstruction, airway hyperresponsiveness, bronchiolitis obliterans organizing pneumonia, bronchiectasis, bronchiolitis, bronchiolitis obliterans, chronic bronchitis, cor pulmonae, dyspnea, emphysema, hypercapnea, hyperinflation, hypoxemia, hyperoxia-induced inflammations, hypoxia, surgical lung volume reduction, pulmonary fibrosis, pulmonary hypertension, right ventricular hypertropy, sarcoidosis, small airway disease, ventilation-perfusion mismatching, wheeze, colds and lupus.

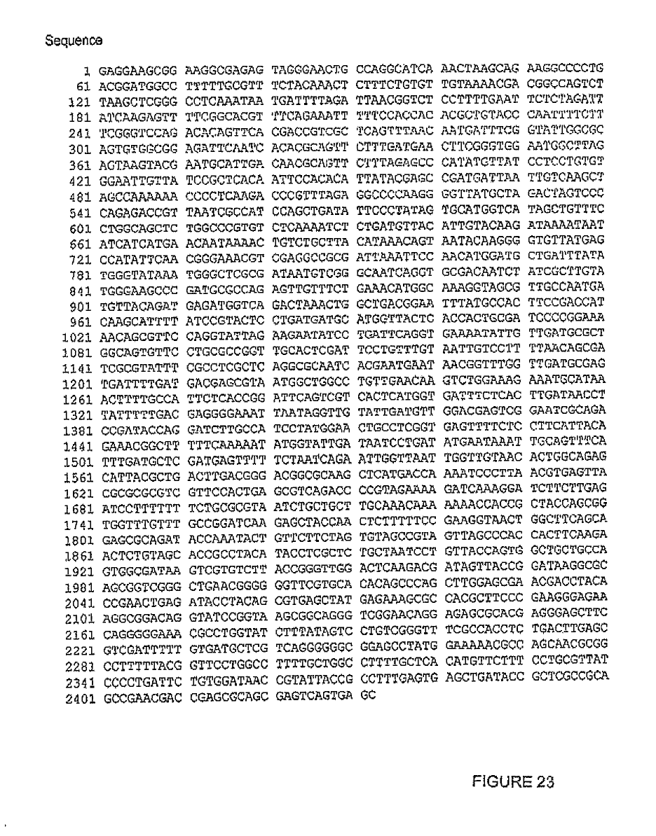

In one embodiment, the promoter is the pTrc promoter (SEQ ID Nos 41 and 43). An example of such a construct is shown in FIG. 23, which shows the nucleotide sequence of the pJ5:G03799 plasmid (SEQ ID No. 3). In another embodiment, there is provided the pJ5:G03799 plasmid having a nucleotide sequence as set forth in SEQ ID No. 3.

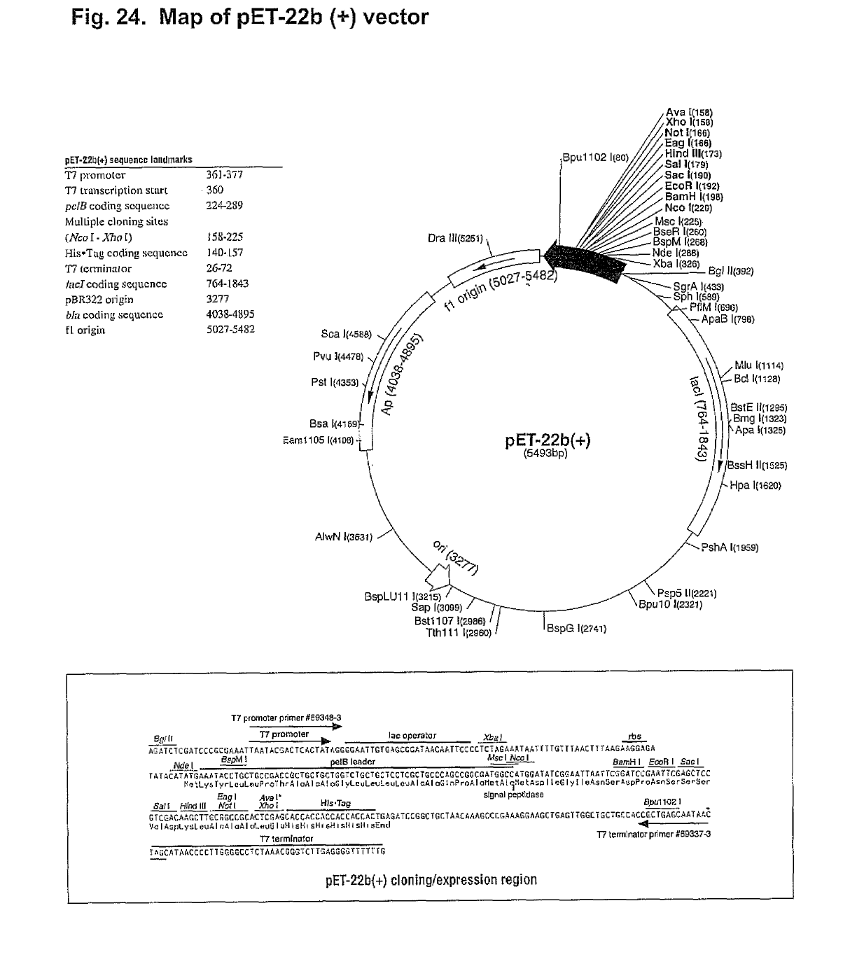

In another embodiment, the promoter is the T7 promoter (SEQ ID No. 42). An example of such a construct is shown in FIG. 24, which shows the general structure of pET-22(b):hG31P⁺².

When amino terminal truncation of bovine CXCL8 is combined with a lysine to arginine substitution at amino acid 11 (i.e., CXCL8₍₃₋₇₄₎K11R), dramatic increases in CXCR1 and CXCR2 receptor affinity are evident, such that CXCL8₍₃₋₇₄₎K11R competitively inhibits the binding of multiple ligands to both receptors (Li, F., and J. R. Gordon. 2001. Biochem. Biophys. Res. Comm. 286:595-600, hereby incorporated by reference). Further truncation into the receptor-signaling ELR motif (e.g., amino acids 4-6 of human CXCL8) of some CXC chemokines can transform them into mild (CXCL8₍₆₋₇₂₎) to moderate (CXCL1₍₈₋₇₃₎) receptor antagonists (McColl and Clark Lewis 1999; Moser, B. et al. 1993 J. Biol. Chem. 268:7125-7128). As disclosed herein, the introduction into bovine CXCL8₍₃₋₇₄₎K11R of a second amino acid substitution, glycine 31 to a proline residue (i.e., CXCL8₍₃₋₇₄₎K11R/G31P), renders this CXCL8 analogue a very high affinity antagonist of bovine and human ELR-CXC chemokine responses. It fully antagonizes the entire array of ELR-CXC chemokines expressed within bacterial or endotoxin-induced inflammatory foci and blocks endotoxin-induced inflammation in vivo.

G31P constructs discussed herein include:

(Bovine 3-74 K11RG31P - SEQ ID No. 4)

TELRCQCIRTHSTPFHPKFIKELRVIESPPHCENSEIIVKLTNGNEVCLN
PKEKWVQKVVQVFVKRAEKQDP

(bovine 3-74 K11R P32G - SEQ ID No. 5)

TELRCQCIRTHSTPFHPKFIKELRVIESGGHCENSEIIVKLTNGNEVCLN
PKEKWVQKVVQVFVKRAEKQDP

(bovine - 3-74 T12SH13PG31P - SEQ ID No. 6)

TELRCQCIRSPSTPFHPKFIKELRVIESPPHCENSEIIVKLTNGNEVCLN
PKEKWVQKVVQVFVKRAEKQDP

(bovine - 3-74 T12SH13PG31PP32G - SEQ ID No. 7)

TELRCQCIRSPSTPFHPKFIKELRVIESPGHCENSEIIVKLTNGNEVCLN
PKEKWVQKVVQVFVKRAEKQDP

(bhG31P - SEQ ID No. 8)

GSTELRCQCIRTHSTPFHPKFIKELRVIESPPHCENSEIIVKLTDGRELC
LDPKENWVQRVVEKFLKRAENS;

(hG31P (0) - SEQ ID No. 9)

KELRCQCIRTYSKPFHPKFIKELRVIESPPHCANTEIIVKLSDGRELCLD
PKENWVQRVVEKFLKRAENS;

(hG31P (−1)- SEQ ID No. 10)

ELRCQCIRTYSKPFHPKFIKELRVIESPPHCANTEIIVKLSDGRELCLDP
KENWVQRVVEKFLKRAENS;

(hG31P (+2) - SEQ ID No. 11)

GSKELRCQCIRTYSKPFHPKFIKELRVIESPPHCANTEIIVKLSDGRELC
LDPKRNWVQRVVEKFLKRAENS;

(hG31P (+6) - SEQ ID No. 12)

GSMGGSKELRCQCIRTYSKPFHPKFIKELRVIESPPHCANTEIIVKLSDG
RELCLDPKENWVQRVVEKFLKRAENS;

(hG31P K3T - SEQ ID No. 13)

GSTELRCQCIRTYSKPFHPKFIKELRVIESPPHCANTEIIVKLSDGRELC
LDPKENWVQRVVEKFLKRAENS;

(hG31P Y13H - SEQ ID No. 14)

GSKELRCQCIRTHSRPFHPKFIKELRVIESPPHCANTEIIVKLSDGRELC
LDPKENWVQRVVEKFLKRAENS;

(hG31P K15T (0) - SEQ ID No. 15)

GSKELRCQCIRTYSTPFHPKFIKELRVIESPPHCANTEIIVKLSDGRELC
LDPKENWVQRVVEKFLKRAENS;

(hG31P A35E(0) - SEQ ID No. 16)

GSKELRCQCIRTYSKPFHPKFIKELRVIESPPHCENTEIIVKLSDGRELC
LDPKENWVQRVVEKFLKRAENS;

(hG31P T37S (0) - SEQ ID No. 17)

GSKELRCQCIRTYSKPFHPKFIKELRVIESPPHCANSEIIVKLSDGRELC
LDPKENWVQRVVEKFLKRAENS;

(hG31P S44T (0) - SEQ ID No. 18)

GSKELRCQCIRTYSKPFHPKFIKELRVIESPPHCANTEIIVKLTDGRELC
LDPKENWVQRVVEKFLKRAENS;

(hG31P R47D (0) - SEQ ID No. 19)

GSKELRCQCIRTYSKPFHPKFIKELRVIESPPHCANTEIIVKLSDGDELC
LDPKENWVQRVVEKFLKRAENS;

(hG31P N56K(0) - SEQ ID No. 20)

GSKELRCQCIRTYSKPFHPKFIKELRVIESPPHCANTEIIVKLSDGRELC
LDPKEKWVQRVVEKFLKRAENS;

(hG31P R60K (0) - SEQ ID No. 21)

GSKELRCQCIRTYSKPFHPKFIKELRVIESPPHCANTEIIVKLSDGRELC
LDPKENWVQKVVEKFLKRAENS;

(hG31P K64V (0) - SEQ ID No. 22)

GSKELRCQCIRTYSKPFHPKFIKELRVIESPPHCANTEIIVKLSDGRELC
LDPKENWVQRVVEVFLKRAENS;

(hG31P L49V - SEQ ID No. 23)

GSKELRCQCIRTYSKPFHPKFIKELRVIESPPHCANTEIIVKLSDGREVC
LDPKENWVQRVVEKFLKRAENS;

(bhG31P T3K - SEQ ID No. 24)

GSKELRCQCIRTHSTPFHPKFIKELRVIESPPHCENSEIIVKLTDGRELC
LDPKENWVQRVVEKFLKRAENS;

(bhG31P H13Y - SEQ ID No. 25)

GSTELRCQCIRTYSTPFHPRFIKELRVIESPPHCENSEIIVKLTDGRELC
LDPKYNWVQRVVEKFLRRAENS;

(bhG31P T15K - SEQ ID No. 26)

GSTELRCQCIRTHSKPFHPKFIKELRVIESPPHCENSEIIVKLTDGRELC
LDPKENWVQRVVEKFLKRAENS;

(bhG31P E35A - SEQ ID No. 27)

GSTELRCQCIRTHSTPFHPKFIKELRVIESPPHCANSEIIVKLTDGRELC
LDPKENWVQRVVEKFLKRAENS;

(bhG31P S3YT - SEQ ID No. 28)

GSTELRCQCIRTHSTPFHPKFIKELRVIESPPHCENTEIIVKLTDGRELC
LDPKENWVQRVVEKFLKRAENS;

(bhG31P (+6) - SEQ ID No. 29)

GSMGGSTELRCQCIRTHSTPFHPKFIKELRVIESPPHCENSEIIVKLTDG
RELCLDPKENWVQRVVEKFLKRAENS;

(bhG31P (+2) - SEQ ID No. 30)

GSTELRCQCIRTHSTPFHPKFIKELRVIESPPHCENSEIIVKLTDGRELC
LDPKENWVQRVVEKFLKRAENS;

(bhG31P (+0) - SEQ ID No. 31)

TELRCQCIRTHSTPFHPKFIKELRVIESPPHCENSEIIVKLTDGRELCLD
PKENWVQRVVEKFLKRAENS;

(bhG31P (−1) - SEQ ID No. 32)

ELRCQCIRTHSTPFHPKFIKELRVIESPPHCENSEIIVKLTDGRELCLDP
KENWVQRVVEKFLKRAENS;

(hK11R (+6) - SEQ ID No. 33)

GSMGGSKFLRCQCIRTYSKPFHPKFIKELRVIESGPHCANTEIIVKLSDG
RELCLDPKENWVQRVVEKFLKRAENS;

(hK11RG31P A35E(+6) - SEQ ID No. 34)

GSMGGSKELRCQCIRTYSKPFHPKFIKELRVIESPPHCENTEIIVKLSDG
RELCLDPKENWVQRVVEKFLKRAENS;

hK11RG31P T37S (+6) - SEQ ID No. 35)

GSMGGSKELRCQCIRTYSKPFHPKFIKELRVIESPPHCANSEIIVKLSDG
RELCLDPKENWVQRVVEKFLKRAENS;

(hK11RG31P S44T (+6) - SEQ ID No. 36)

GSMGGSKELRCQCIRTYSKPFHPKFIKELRVIESPPHCANTEIIVKLTDG
RELCLDPKENWYQRVVEKFLKRAENS;

(hK11RG31P R47D (+6) - SEQ ID No. 37)

GSMGGSKELRCQCIRTYSKPFHPKFIKELRVIESPPHCANTEIIVKLSDG
DELCLDPKENWVQRVVEKFLKRAENS;

(hK11RG31P L49V - SEQ ID No. 38)

GSMGGSKELRCQCIRTYSKPFHPKFIKELRVIESPPHCANTEIIVKLSDG
REVCLDPKENWVQRVVEKFLKRAENS;
and

(Bovine 3-74 K11RG31P (+2) - SEQ ID No. 39)

GSTELRCQCIRTHSTPFHPKFIKELRVIESPPHCENSEIIVKLTNGNEVC
LNPKEKWVQKVVQVFVKRAEKQDP.

### Material and Methods

Reagents & supplies. The following reagents were purchased commercially: glutathione-Sepharose, the expression vector pGEX-2T, Sephadex G-25 (Amersham-Pharmacia-Biotech, Baie d'Urf, PQ), Bolton-Hunter reagent, a protein biotinylation kit (Pierce Scientific, Rockford, Ill.), the sequencing vector pBluescript II KS, Pfu Turbo™ DNA polymerase (Stratagene, La Jolla, Calif.), a site-directed mutagenesis kit (QuickChange™; Boerhinger-Mannheim Canada, Laval, PQ), aprotinin, benzene, calcium ionophore A23187, chloramine T, cytochalasin B, dimethylformamide, endotoxin (Escherichia coli lipopolysaccharide, serotype 0127B8), isopropyl-thio-D-galactopyranoside (IPTG), leupeptin, p-nitrophenyl-D-glucuronide, mineral oil, silicon oil, tetramethylbenzidine (TMB), phenylmethylsulfonyl fluoride (PMSF), phorbol-12,13-myristate acetate (PMA), and Triton X-100 (Sigma Chemical Co, Mississauga, ON), a Diff-Quick staining kit (American Scientific Products, McGaw Pk, Ill.), human CXCL1, CXCL5, and CXCL8 (R & D Systems Inc, Minneapolis, Minn.), horse radish peroxidase (HRP)-conjugated anti-rabbit Ig (Zymed, South San Francisco, Calif.), DMEM, HBSS (Gibco, Grand Island, N.Y.), HRP-streptavidin (Vector Labs, Burlingame, Calif.), ABTS enzyme substrate (Kirkegaard & Perry Labs, Gaithersburg, Md.), bovine serum albumin (BSA), and Lymphocyte Separation Medium (ICN Pharmaceuticals, Aurora, Ill.).

Generation of CXCL8₍₃₋₇₄₎K11R analogues. The high affinity CXCR1/CXCR2 ligand CXCL8₍₃₋₇₄₎K11R, and its T12S/H₁₃F analogue were generated in accordance with the methods described in Li and Gordon (2001, supra). The Gly31Pro (G31P), Pro32Gly (P32G), and G31P/P32G analogues of these proteins were similarly generated by site-directed mutagenesis using PCR with the appropriate forward and reverse oligonucleotide primers (Table 1). The products from each reaction were digested with DpnI, ligated into the vector pGEX-2T, transfected into HB101 cells, and their sequences verified commercially (Plant Biotechnology Institute, Saskatoon). Briefly, the recombinant bacteria were lysed in the presence of a protease inhibitor cocktail (2 mM PMSF, 2 μg/ml aprotinin, and 2 μg/ml leupeptin) and the recombinant fusion proteins in the supernatants purified by affinity chromatography, using glutathione-Sepharose beads in accordance with the methods of Caswell et al. (Caswell, J. L., D. M. Middleton, and J. R. Gordon. 1998. Vet. Immunol. Immunopath. 67:327-340.). The CXCL8₍₃₋₇₄₎K11R analogues were cleaved from the GST fusion proteins by thrombin digestion, dialysed against phosphate buffered saline (PBS), run through commercial endotoxin-removal columns, and then characterized by polyacrylamide gel electrophoresis (PAGE) and Western blotting with a goat anti-bovine CXCL8 antibody (provided by Dr. M. Morsey). Each purified analogue had a molecular mass of 8 kDa, was specifically recognized by the anti-CXCL8 antibody in the Western blotting, and had a relative purity of 96%, as determined by densitometric analysis of the PAGE gels.

Labeling of the recombinant proteins. We used ^{biot}CXCL8 for the initial surveys of analogue binding to neutrophils and ¹²⁵I-CXCL8 for the later stage assays of relative receptor affinity. CXCL8 was biotinylated and the levels of biotin substitution determined using a commercial kit, as noted in Li and Gordon (2001, supra). The ^{biot}CXCL8 was substituted with 2.15 moles of biotin per mole of CXCL8. CXCL8 was radiolabeled with ¹²⁵I using the Bolton-Hunter Reagent (BHR) method, as noted in detail (Li and Gordon 2001, supra). The labeled protein was separated from the unincorporated ¹²⁵I-BHR by chromatography on Sephadex G50, and the labeled CXCL8 characterized for its relative affinity for neutrophils and the time required to achieve binding equilibrium, as noted in Li and Gordon (2001, supra).

CXCL8₍₃₋₇₄₎K11R analogue binding assays. Cells (85-93% neutrophils) were purified from the blood of cattle in accordance with the Caswell method (Caswell, J. L. et al. 1998. Vet. Immunol. Immunopath. 67:327-340). In preliminary experiments, we determined that none of our analogues affected the viability of neutrophils, as determined by trypan blue dye exclusion. For the broad analogue surveys, neutrophils in HBSS/0.5% BSA were incubated for 2 h at 4° C. with the analogue, washed in cold DMEM, and then incubated for another 2 h at 4° C. with ^{biot}CXCL8 (1000 ng/ml). The cell-associated biotin was detected by incubating the washed cells with alkaline phosphatase-conjugated streptavidin (1:700 dilution) and then with ABTS enzyme substrate. The OD₄₀₅ of the samples was determined using an ELISA plate reader. Medium-treated neutrophils routinely bound sufficient ^{biot}CXCL8 to generate an OD₄₀₅ of 0.5-0.6.

For the in-depth studies with CXCL8₍₃₋₇₄₎K11R/G31P, we used ¹²⁵I-CXCL8 in binding inhibition assays with unlabeled CXCL8 or CXCL8₍₃₋₇₄₎K11R/G31P. In preliminary experiments we determined that the binding equilibrium time of neutrophils for ¹²⁵I-CXCL8 was 45 min and that 20 pM ¹²⁵I-CXCL8 just saturated the cell's high affinity receptors. Thus, in our assays, 10⁶ purified neutrophils were incubated for 45 min on ice with 20 pM ¹²⁵I-CXCL8 and varying concentrations of unlabeled competitor ligand. The cells were then sedimented through 6% mineral oil in silicon oil and the levels of cell-associated radio-ligand determined using a counter. The non-specific binding of ¹²⁵I-CXCL8 to the cells was assessed in each assay by including a 200-fold molar excess of unlabeled ligand in a set of samples. This value was used to calculate the percent specific binding (Coligan, J., A. Kruisbeek, D. Margulies, E. Shevach, and W. Strober. 1994. Current Protocols in Immunology. John Wiley & Sons, New York).

Neutrophil-glucuronidase release assay. The neutrophil-glucuronidase assay has been reported in detail (Li and Gordon 2001, supra). Briefly, cytochalasin B-treated neutrophils were incubated for 30 min with the CXCL8 analogues, then their secretion products assayed calorimetrically for the enzyme.-Glucuronidase release was expressed as the percent of the total cellular content, determined by lysing medium-treated cells with 0.2% (v/v) Triton X-100. Neutrophil challenge with the positive control stimulus PMA (50 ng/ml) and A23187 (1 μg/ml) induced 42+/−6% release of the total cellular-glucuronidase stores.

Samples from inflammatory lesions. We obtained bronchoalveolar lavage fluids (BALF) from the lungs of cattle (n=4) with diagnosed clinical fibrinopurulent pneumonic mannheimiosis (Caswell et al., 1997), as well as teat cistern wash fluids from cattle (n=4) with experimental endotoxin-induced mastitis (Waller, K. P. 1997. Vet. Immunol. Immunopathol. 57:239-251). In preliminary dose-response experiments we determined that 5 μg of endotoxin induced a strong (70-80% maximal) mammary neutrophil response. Thus, in the reported experiments mastitis was induced by infusion of 5 μg of endotoxin or carrier medium alone (saline; 3 ml volumes) into the teat cisterns of non-lactating Holstein dairy cows, and 15 h later the infiltrates were recovered-from the cisterns by lavage with 30 ml HBSS. The cells from the BALF and teat cistern wash fluids were sedimented by centrifugation and differential counts performed. Untreated and CXCL8-depleted (below) wash fluids were assessed for their chemokine content by ELISA (CXCL8 only) and chemotaxis assays.

Neutrophil chemotaxis assays. Microchemotaxis assays were run in duplicate modified Boyden microchemotaxis chambers using polyvinylpyrrolidone-free 5 μm pore-size polycarbonate filters, in accordance with known methods (Caswell et al., 1998; Cairns, C. M. et al. 2001. J. Immunol. 167:57-65). For each sample, the numbers of cells that had migrated into the membranes over 20-30 min were enumerated by direct counting of at least nine 40×. objective fields, and the results expressed as the mean number of cells/40× field (+/−SEM). The chemoattractants included bovine or human CXCL8, human CXCL5 and CXCL1, pneumonic mannheimiosis BALF and mastitis lavage fluids (diluted 1:10-1:80 in HBSS), while the antagonists comprised mouse anti-ovine CXCL8 antibody 8M6 (generously provided by Dr. P. Wood, CSIRO, Australia) or the CXCL8₍₃₋₇₄₎K11R analogues. In some assays we preincubated the samples with the antibodies (5 μg/ml) for 60 min on ice (Gordon, J. R. 2000. Cell Immunol. 201:42-49). In others we generated CXCL8-specific immunoaffinity matrices with the 8M6 antibodies and protein-A-Sepharose beads and used these in excess to absorb the samples (Caswell et al., 1997; Gordon, J. R., and S. J. Galli. 1994. J. Exp. Med. 180:2027-2037); the extent of CXCL8 depletion was confirmed by ELISA of the treated samples. For assays with the recombinant antagonists, the inhibitors were mixed directly with the samples immediately prior to testing.

CXCL8 ELISA. For our ELISA, MAb 8M6 was used as the capture antibody, rabbit antiovine CXCL8 antiserum (also from P. Wood, CSIRO) as the secondary antibody, and HRP-conjugated anti-rabbit Ig, and TMB as the detection system, as noted in Caswell et al. (1997). Serial dilutions of each sample were assayed in triplicate, and each assay included a recombinant bovine CXCL8 standard curve.

CXCL8₍₃₋₇₄₎K11R/G31P blockade of endotoxin responses in vivo. We used a sequential series of 15 h skin tests to test the ability of CXCL8₍₃₋₇₄₎K11R/G31P to block endotoxin induced inflammatory responses in vivo. For each test, we challenged 2 week-old healthy Holstein cows intradermally with 1 μg endotoxin in 100 μl saline, then 15 h later took 6 mm punch biopsies under local anaesthesia (lidocaine) and processed these for histopathology (Gordon and Galli, 1994). Following the first (internal positive control) test, we injected each animal subcutaneously, intramuscularly, or intravenously with CXCL8₍₃₋₇₄₎K11R/G31P (75 μg/kg) in saline, then challenged them again with endotoxin, as above. The animals were challenged a total of 4 times with endotoxin, such that 15 h reaction site biopsies were obtained at 0, 16, 48, and 72 h post-treatment. The biopsies were processed by routine methods to 6 μm paraffin sections, stained with Giemsa solution, and examined in a blinded fashion at 400-magnification (Gordon and Galli, 1994; Gordon, J. R. 2000. J. Allergy Clin. Immunol. 106:110-116). The mean numbers of neutrophils per 40× objective microscope field were determined at three different depths within the skin, the papillary (superficial), intermediate, and reticular (deep) dermis.

Statistical analyses. Multi-group data were analyzed by ANOVA and post-hoc Fisher protected Least Significant Difference (PLSD) testing, while two-group comparisons were made using the students t-test (two-tailed). The results are expressed as the mean+/−SEM.

### Results

### 1. Bovine G31P

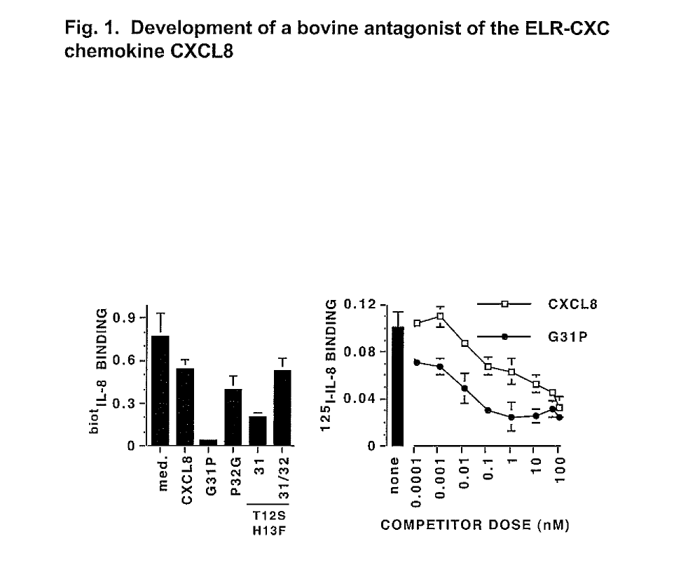

CXCL8₍₃₋₇₄₎K11R/G31P competitively inhibits CXCL8 binding to neutrophils. We surveyed the ability of each CXCL8₍₃₋₇₄₎K11R analogue to bind to the CXCL8 receptors on neutrophils, and thereby compete with CXCL8 as a ligand. In our initial surveys, we employed ^{biot}CXCL8 binding inhibition assays, incubating the cells with the analogues (10 ng/ml) for 2 h at 4° C. prior to exposure to ^{biot}CXCL8 (1 μg/ml). This level of CXCL8 approximates those found in the lung tissues of sheep with experimental pneumonic mannheimiosis (Caswell, J. L. 1998. The role of interleukin-8 as a neutrophil chemoattractant in bovine bronchopneumonia. Ph.D. thesis, Department of Veterinary Pathology, University of Saskatchewan). We found that CXCL8₍₃₋₇₄₎K11R/G31P was a potent antagonist of CXCL8 binding in this assay (FIG. 1), such that 10 ng/ml of CXCL8₍₃₋₇₄₎K11R/G31P blocked 95% of subsequent ^{biot}CXCL8 binding to the cells. When tested at this dose, CXCL8₍₃₋₇₄₎K11R/P32G blocked only 48% of CXCL8 binding, while unlabeled CXCL8 itself competitively inhibited 30% of ^{biot}CXCL8 binding. Introduction into CXCL8₍₃₋₇₄₎K11R/G31P or CXCL8₍₃₋₇₄₎K11R/P32G of additional amino acid substitutions at Thr12 and His13 substantially reduced the antagonist activities of the analogues (FIG. 1). This data clearly suggests that pre-incubation of neutrophils with CXCL8₍₃₋₇₄₎K11R/G31P strongly down-regulates subsequent binding of CXCL8.

In order to more finely map the ability of CXCL8₍₃₋₇₄₎K11R/G31 to inhibit the binding of CXCL8, in our next set of experiments we simultaneously exposed the cells to ¹²⁵I-CXCL8 and varying doses of CXCL8₍₃₋₇₄₎K11R/G31P or unlabeled CXCL8. We found that CXCL8₍₃₋₇₄₎K11R/G31P was about two orders of magnitude more effective than wildtype CXCL8 in inhibiting the binding of 20 pM ¹²⁵I-CXCL8 to the cells (FIG. 1). The concentration for inhibiting 50% of labeled ligand binding (IC₅₀) was 120 pM for unlabelled CXCL8, and 4 pM for CXCL8₍₃₋₇₄₎K11R/G31P. This data suggests that CXCL8₍₃₋₇₄₎K11R/G31P is a very potent competitive inhibitor of CXCL8 binding to neutrophils.

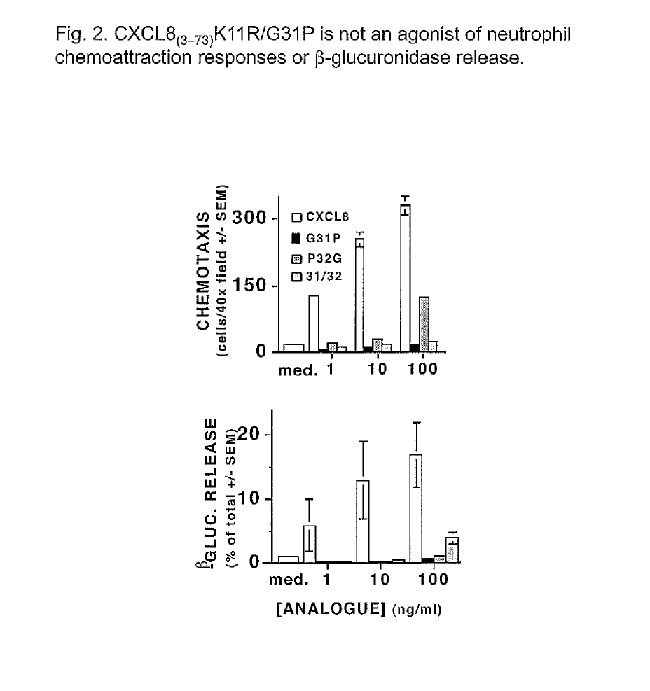

CXCL8₍₃₋₇₄₎K11R/G31P does not display neutrophil agonist activities. While CXCL8₍₃₋₇₄₎K11R/G31P was certainly a high affinity ligand for the neutrophil CXCL8 receptors, it would equally well do so as an agonist or an antagonist. Thus our next experiments addressed the potential agonist activities of the CXCL8₍₃₋₇₄₎K11R analogues we generated, as measured by their abilities to chemoattract these cells or induce release of the neutrophil granule hydrolytic enzyme-glucuronidase in vitro (FIG. 2). We found that even at 100 ng/ml, CXCL8₍₃₋₇₄₎K11R/G31P was a poor chemoattractant, inducing 13.9+/−4% or 5.4+1-2% of the responses induced by 1 or 100 ng/ml CXCL8 (p<0.001), respectively. At 100 ng/ml, the CXCL8₍₃₋₇₄₎K11R/P32G analogue induced a response that was fairly substantial (38.3+/−2% of the CXCL8 response), while the combined CXCL8₍₃₋₇₄₎K11R/G31P/P32G analogue also was not an effective chemoattractant. When we assessed their abilities to induce-glucuronidase release, we found that none of the CXCL8₍₃₋₇₄₎K11R analogues was as effective as CXCL8 in inducing mediator release. Indeed, we found only background release with any of them at 10 ng/ml, and at 100 ng/ml only CXCL8₍₃₋₇₄₎K11R/G31P/P32G induced significant neutrophil responses (FIG. 2). Given the combined CXCL8 competitive inhibition and neutrophil agonist data, from this point on we focused our attention on CXCL8₍₃₋₇₄₎K11R/G31P.

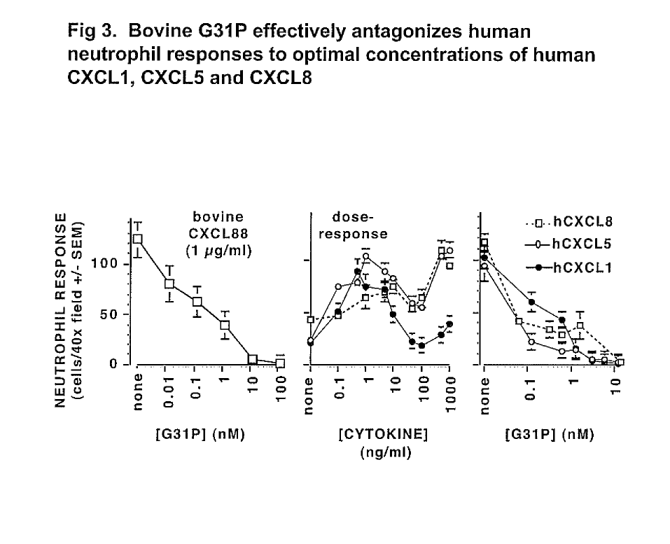

CXCL8₍₃₋₇₄₎K11R/G31P blocks neutrophil chemotactic responses to both CXCR1 and CXCR2 ligands. The most pathogenic effect of inappropriate ELR⁺ chemokine expression is the attraction of inflammatory cells into tissues. Thus, we next assessed the impact of CXCL8₍₃₋₇₄₎K11R/G31P on the chemotactic responses of neutrophils to high doses of CXCL8 (FIG. 3). As predicted from our in vivo observations in sheep and cattle (33), 1 μg/ml (129 nM) CXCL8 was very strongly chemoattractive, but even very low doses of CXCL8₍₃₋₇₄₎K11R/G31P ameliorated this response. The addition of 12.9 pM CXCL8₍₃₋₇₄₎K11R/G31P reduced the chemotactic response of the cells by 33%. The IC₅₀ for CXCL8₍₃₋₇₄₎K11R/G31P under these conditions was 0.11 nM, while complete blocking of this CXCL8 response was achieved with 10 nM CXCL8₍₃₋₇₄₎K11R/G31P.

When we tested the efficacy of CXCL8₍₃₋₇₄₎K11R/G31P in blocking responses to more subtle bovine CXCL8 challenges, we also extended the study to assess the ability of CXCL8₍₃₋₇₄₎K11R/G31P to block neutrophil responses to human CXCL8 as well as to the human CXCR2-specific ligands CXCL1 and CXCL5. Each of these is expressed in the affected tissues of pancreatitis (Hochreiter, W. W. et al. 2000. Urology. 56:1025-1029) or ARDS (Villard et al., 1995) patients at 1-10 ng/ml. We found that bovine neutrophils were responsive to 1 ng/ml hCXCL1 or hCXCL5, and similarly responsive to 10 ng/ml hCXCL8 (FIG. 3), so we employed these doses to test the effects of CXCL8₍₃₋₇₄₎K11R/G31P on neutrophil responses of these ligands. The neutrophil responses to hCXCL1 and hCXCL5 were reduced to 50% by 0.26 and 0.06 nM CXCL8₍₃₋₇₄₎K11R/G31P, respectively, while their responses to hCXCL8 were 50% reduced by 0.04 nM CXCL8₍₃₋₇₄₎K11R/G31P (FIG. 3). This data indicates that CXCL8₍₃₋₇₄₎K11R/G31P can antagonize the actions of multiple members of the ELR-CXC subfamily of chemokines.

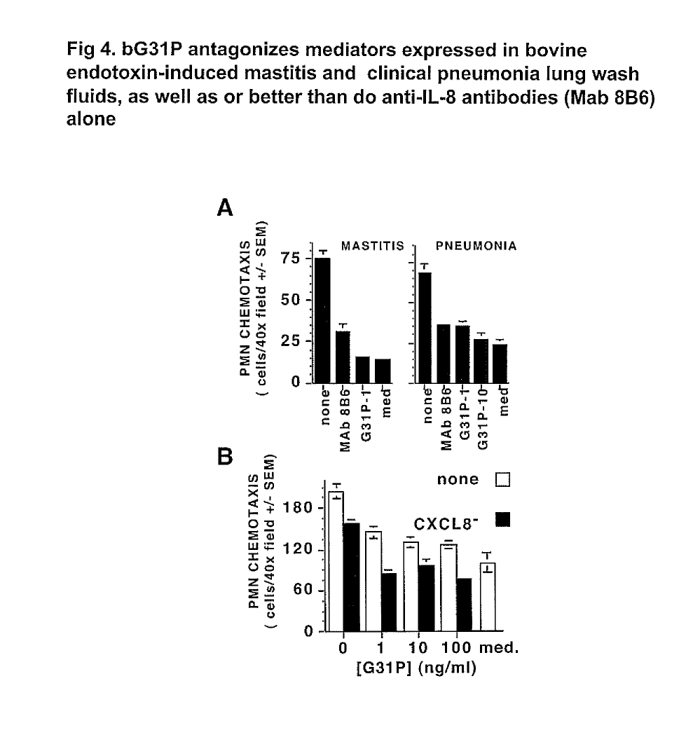

CXCL8₍₃₋₇₄₎K11R/G31P is an effective in vitro antagonist of the neutrophil chemokines expressed in bacterial pneumonia or mastitis lesions. We wished to test the extent to which our antagonist could block the array of neutrophil chemoattractants expressed within complex inflammatory environments in vivo. Thus, we chose two diseases in which chemokine-driven neutrophil activation contributes importantly to the progression of the pathology, mastitis and pneumonic mannheimiosis. We utilized an endotoxin model of mastitis (Persson, K. et al., 1993. Vet. Immunol. Immunopathol. 37:99-112), in which we infused 5 μg of endotoxin/teat cistern and 15 h later lavaged each cistern. Neutrophils comprised 82 and 6%, respectively, of the cells from endotoxin and saline-control cisterns, with the bulk of the remaining cells comprising macrophages. The diluted (1:10) wash fluids induced strong in vitro neutrophil chemotactic responses, and the addition of anti-CXCL8 antibodies to the samples maximally reduced these by 73+/−8% (FIG. 4A), relative to the medium control. On the other hand, the addition of 1 ng/ml of CXCL8₍₃₋₇₄₎K11R/G31P to the samples reduced their chemotactic activity by 97+/−3%.

Neutrophils also comprised 93+/−12% of the cells recovered from the BALF of cattle with advanced pneumonic mannheimiosis. When tested in vitro, these samples too were strongly chemotactic for neutrophils, and the addition of anti-CXCL8 antibodies maximally reduced their neutrophil chemotactic activities by 73+/−5% (FIG. 4A). Treatment of these BALF samples with 1 or 10 ng/ml of CXCL8₍₃₋₇₄₎K11R/G31P reduced the neutrophil responses by 75+/−9 or 93+/−9%, respectively, relative to the medium controls. This data suggests that CXCL8₍₃₋₇₄₎K11R/G31P blocks the actions of CXCL8 and non-CXCL8 chemoattractants in these samples.

In order to confirm these observations using an alternate strategy, we next depleted bacterial pneumonia BALF samples of CXCL8 using immunoaffinity matrices, then assessed the efficacy of CXCL8₍₃₋₇₄₎K11R/G31P in blocking the residual neutrophil chemotactic activities in the samples (FIG. 4B). The untreated lesional BALF samples contained 3,215+/−275 pg/ml CXCL8, while the immunoaffinity-absorbed BALF contained 24+/−17 pg/ml CXCL8. In this series of experiments the neutrophil response to the CXCL8-depleted BALF samples was 65.4+1-4% of their responses to the unabsorbed samples. It is known that CXCL8 can contribute as little as 15% of the neutrophil chemotactic activities in pneumonic mannheimiosis BALF obtained from an array of clinical cases (Caswell et al., 2001). Whereas the CXCL8 depletion treatments were 99% effective in removing CXCL8, there remained in these samples substantial amounts of neutrophil chemotactic activities, and the addition of 1 ng/ml CXCL8₍₃₋₇₄₎K11R/G31P fully abrogated their cumulative effects (FIG. 4B). This data unequivocally confirmed that CXCL8₍₃₋₇₄₎K11R/G31P also antagonizes the spectrum of non-IL-8 chemoattractants expressed in these samples.

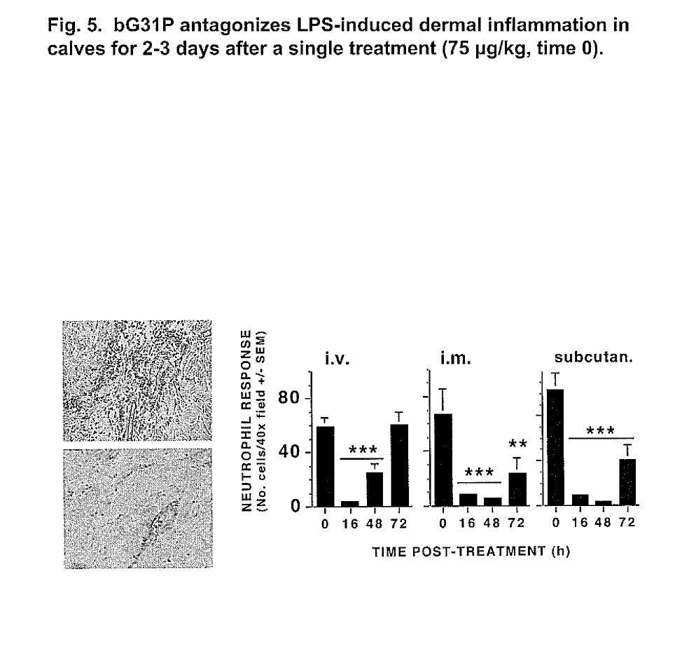

CXCL8₍₃₋₇₄₎K11R/G31P is highly efficacious in blocking endotoxin-induced neutrophilic inflammation in vivo. In our last experiments, we assessed the ability of CXCL8₍₃₋₇₄₎K11R/G31P to block endotoxin-induced inflammatory responses in the skin of cattle, as well as the time-frames over which it was effective. The animals were challenged intradermally with 1 μg bacterial endotoxin 15 h before (internal positive control response), or at three different times after, intravenous, subcutaneous or intramuscular injection of CXCL8₍₃₋₇₄₎K11R/G31P (75 μg/kg). Thus, punch biopsies of 15 h endotoxin reaction sites were taken 15 min before treatment and at 16, 48 and 72 h after injection of the antagonist into each animal, and the numbers of infiltrating neutrophils were determined in a blinded fashion for the papillary (superficial), intermediate and reticular dermis of each biopsy. Prior to the antagonist treatments, strong neutrophilic inflammatory responses were evident at the endotoxin challenge sites in each animal (FIG. 5). Within the biopsies, the responses in the papillary dermis were mild in all animals (data not shown) and became progressively more marked with increasing skin depth, such that maximal inflammation (neutrophil infiltration) was observed around the blood vessels in the reticular dermis (FIG. 5A). Following the CXCL8₍₃₋₇₄₎K11R/G31P treatments, the inflammatory responses observed within the 16 h biopsies were 88-93% suppressed, while those in the 48 h biopsies were 57% (intravenous) to 97% (intradermal) suppressed, relative to their respective pretreatment responses. By 72 h post-treatment the effects of the intravenously administered antagonist had worn off, while the endotoxin responses in the intradermally and subcutaneously treated cattle were still 60% suppressed. This data clearly indicates that CXCL8₍₃₋₇₄₎K11R/G31P is a highly effective antagonist of endotoxin-induced inflammatory responses in vivo, that these effects can last for 2-3 days, and that the route of delivery markedly affects the pharmacokinetics of this novel antagonist.

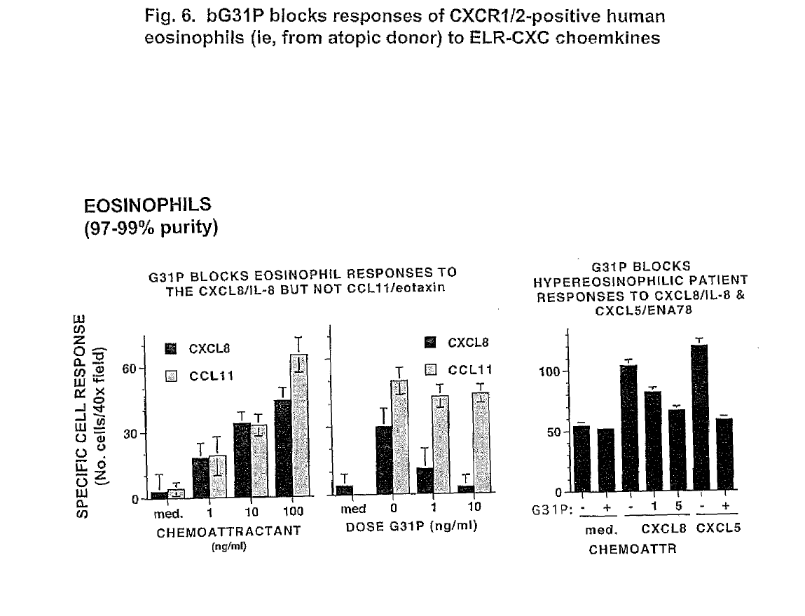

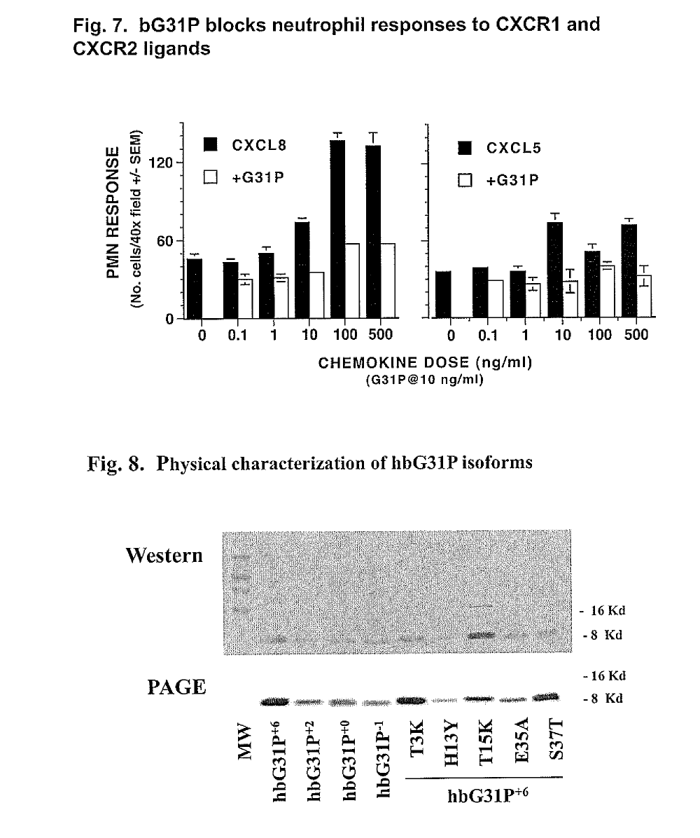

We have found that G31P antagonizes also the chemotactic effects of the human ELR-CXC chemokines CXCL8/IL-8 and CXCL5/ENA-78 on human neutrophils. Thus, the chemotactic activities of 0.1 to 500 ng/ml of either CXCL8 (FIG. 6, left panel) or CXCL5/ENA-78 (FIG. 6, right panel) were essentially completely blocked by the addition of 10 ng/ml of our antagonist to the chemotaxis assays. Similarly, G31P blocked the chemotactic effects of CXCL8 for CXCR1/CXCR2-positive eosinophils. We and others have found that eosinophils from atopic or asthmatic subjects express both ELR-CXC chemokine receptors, and are responsive to CXCL8 (FIG. 7, left panel). The chemotactic effects of 100 ng/ml CXCL8, but not the CCR3 ligand CCL11/eotaxin, on purified peripheral blood eosinophils of an mildly atopic, non-asthmatic donor (99% purity) were completely abrogated by the addition of 10 ng/ml G31P to the chemotaxis assays (FIG. 7, middle panel). When tested against purified eosinophils from a hypereosinophilic patient (FIG. 7, right panel), G31P was neutralized the responses of these cells to either CXCL8/IL-8 or CXCL5/ENA-78.

This data clearly indicates that bovine G31P is an effective antagonist of the bovine ELR-CXC chemokines expressed in vivo in response to endotoxin challenge, but also can fully antagonize neutrophil and eosinophil ELR-CXC chemokine receptor responses to CXCL8 and CXCL5, known ligands for both the CXCR1 and CXCR2.

### Humanized Bovine G31P.

We generated a bovine-human chaemeric protein, comprising the amino terminal half of bovine G31P and the carboxy terminal half of human CXCL8 (bhG31P) (SEQ ID No. 8), and found that it has strong neutrophil antagonist activity in vitro and in vivo; indeed, bhG31P may have greater activity than bG31P, or the human forms of G31P. We also generated and characterized 5 alternate forms of bhG31P (SEQ ID No. 24-28) in which human amino acids were substituted for the remaining bovine amino acids—none of these augmented the antagonist activity of the analogues, and some evidence suggests that they may reduce the antagonist activity.

As one approach in generating a human drug, we undertook the humanization of bG31P. Furthermore, since the amino terminal ‘half’ of CXCL8 is more important for CXCL8's biological activity than the carboxy end, and because the carboxy terminal ‘half’ of CXCL8 contains 10 of the molecule's 15 bovine-human discrepant amino acids, we first examined whether wholesale ligation of the carboxy end of hCXCL8 (i.e., encoding amino acids 45-72) onto the amino half of bG31P (i.e., encoding amino acids 3-44) would affect its activity. Specifically, the amino half of CXCL8 is known to carry the critical receptor recognition and signalling motifs and their associated scaffolding structures.

We thus generated the chaemeric bovine-human G31P protein, bCXCL8(3-44)K11R/G31P-hCXCL8(45-72) (bhG31P) (SEQ ID No. 8), then used the cDNA for this protein as a template for substitution of the remaining bovine-human discrepant amino acid residues one-by-one, as discussed above.

We expressed and purified each construct using SOP for enterokinase (bhG31P⁺⁰ & bhG31P⁻¹ isoforms only) or thrombin (all other analogues) cleavage, and characterized them by SDS-PAGE and Western blotting (FIG. 8). Each isoform was ≈8 kD in size, although the bhG31P/T15K (SEQ ID No. 26) and bhG31P/S37T (SEQ ID No. 28) isoform solutions appeared to contain what could be interpreted as low levels of analogue dimers, formed perhaps as a result of high concentrations of protein in the samples or alternately perhaps related to perturbation of those portions of these G31P analogues associated with dimerization. Several amino acids in this region have been reported previously to significantly affect dimerization of human CXCL8.

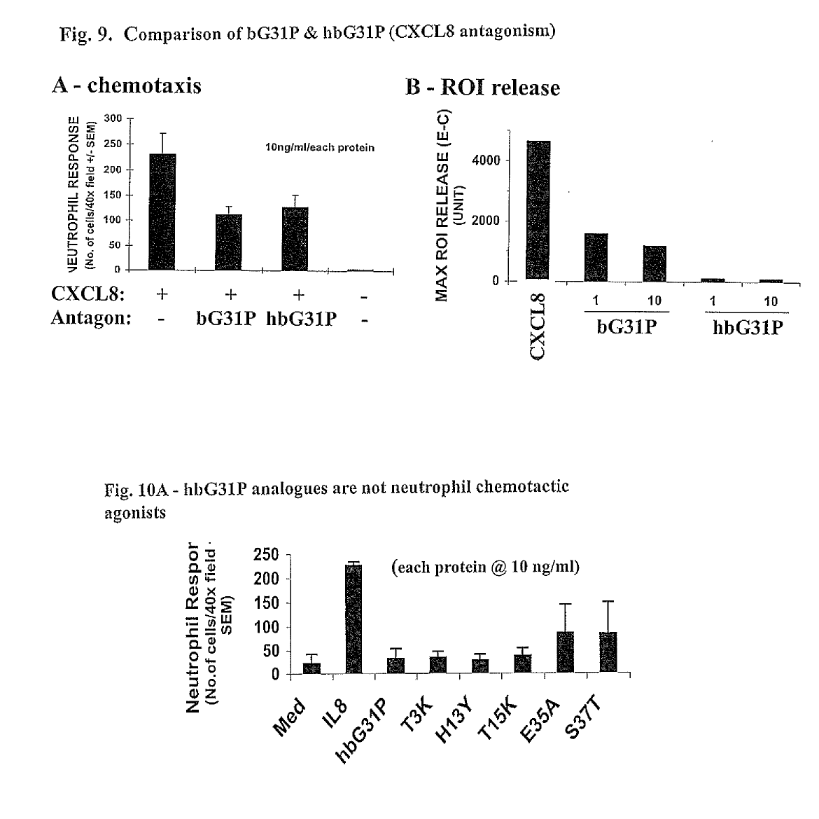

We found that bhG31P (i.e., bCXCL8(3-44)K11R/G31P-hCXCL8(45-72)) retained the ELR-CXC chemokine antagonist activity of bG31P, such that it blocked the chemotactic or reactive oxygen intermediate (ROI) release responses of human neutrophils to human CXCL8 (FIG. 9).

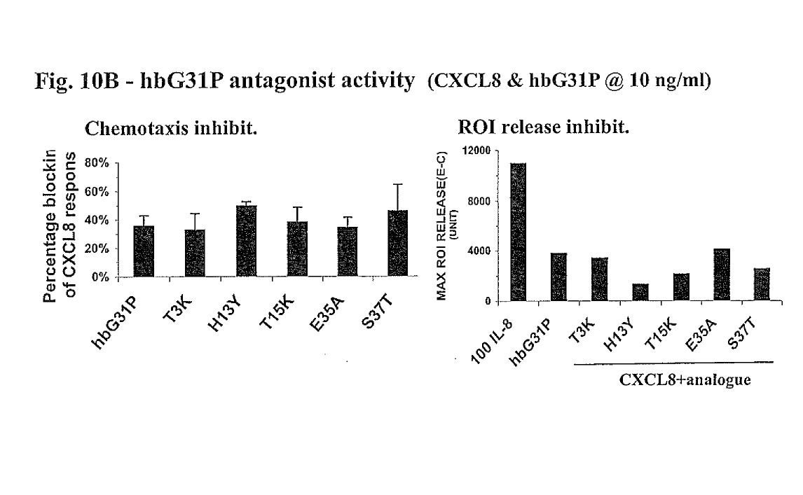

When we further humanized this bovine-human chaemeric protein by introducing additional human-equivalent amino acids in place of the discrepant residues (ie, T3K (SEQ ID No. 24), H13Y (SEQ ID No. 25), T15K (SEQ ID No. 26), E35A (SEQ ID No. 27), and S37T (SEQ ID No. 28)), we found that these changes really had no significant effect on the activity of the 50:50 bh chaemera, neither rendering any of the analogues agonistic for neutrophils as assessed in chemotaxis assays (FIG. 10A) or in terms of significantly reducing the antagonistic activity as assessed by chemotaxis inhibition or by inhibition of reactive oxygen intermediate release (FIG. 10B).

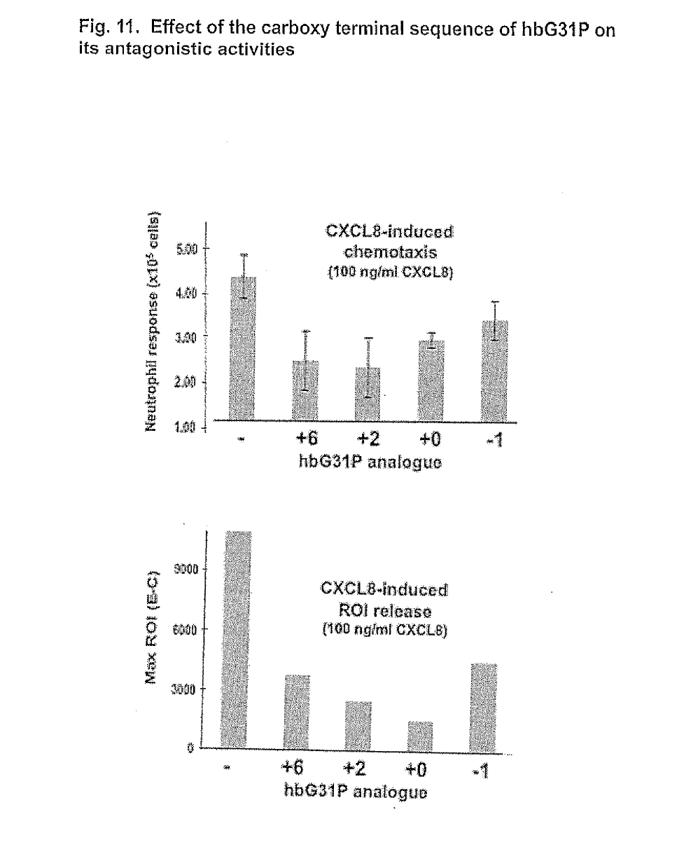

As noted, we assessed the impact on the activity of bhG31P of varying the amino terminal sequence (ie, bhG31P⁺⁶, bhG31P⁺², bhG31P⁺⁰, bhG31P⁻¹) (SEQ ID Nos. 12, 11, 9 and 10 respectively), measuring the activity of each in chemotaxis inhibition and reactive oxygen intermediate inhibition assays (FIG. 11). While we found that eliminating Lys3 tended to reduce the activity of bhG31P, the antagonist activities of the other analogues were roughly equivalent (FIG. 11). As will be appreciated by one of skill in the art, this data indicates that N-terminal additions of varying length and varying amino acid composition would be tolerated without significant disruption of G31P activity. According, N-terminal additions of 0-10 random amino acids or 0-9 random amino acids or 0-8 random amino acids or 0-7 random amino acids or 0-6 random amino acids or 0-5 random amino acids or 0-4 random amino acids or 0-3 random amino acids or 0-2 random amino acids.

Taken together, this data suggests that multiple bovine-human chaemeric forms of G31P are serviceable neutrophil antagonists. It should be noted however, that at about the time we were completing the characterization of these chaemeras, we determined that human forms of CXCL8₍₃₋₇₂₎K11R/G31P were as effective as bG31P in blocking CXCL8-driven neutrophil responses, and that hG31P was also a highly effective antagonist of bacterial endotoxin-driven neutrophilic inflammation and pathology in vivo. Thus, at this point in time we moved most of our efforts to more fully characterizing our hG31P constructs.

3. Human G31P (hG31P).

We contracted with Takara Biotechnology Co., Dalian, PRC to synthesize a full-length human CXCL8 cDNA, which we cloned into pGEX-2T using compatible 5′ (BamH1) and 3′ (EcoR1) ends. This pGEX-hCXCL8 cDNA was used as a template for site-directed mutagenesis to generate pGEX-hCXCL8₍₃₋₇₂₎K11R (hK11R) and pGEX-hCXCL8₍₃₋₇₂₎K11R/G31P (hG31P), which were expressed as GST fusion proteins and purified by thrombin cleavage using standard operating procedures. As with bhG31P (above), we generated two families of recombinant G31P-related molecules that were preceded by either six (Gly-Ser-Met-Gly-Gly-Ser) or two (Gly-Ser) extraneous amino acids, referred to as hG31P⁺⁶ (SEQ ID No. 12) or hG31P⁺² (SEQ ID No. 11), but also additional families of constructs with no exogenous amino acids (G31P⁺⁰) (SEQ ID No. 9) or that were further amino terminal-deleted (ie, G31P⁻¹, G31P⁻³, or G31P⁻⁵). We also introduced into hG31P⁺⁶ the bovine equivalent amino acids at amino acid positions 35 (SEQ ID No. 34), 37 (SEQ ID No. 35), 44 (SEQ ID No. 36), 47 (SEQ ID No. 37) and 49 (SEQ ID No. 38). Each protein was expressed, purified and characterized by SDS-PAGE and Western blotting. As predicted, each comprised a single band of ˜8 kD that was reactive with anti-CXCL8 antibodies.

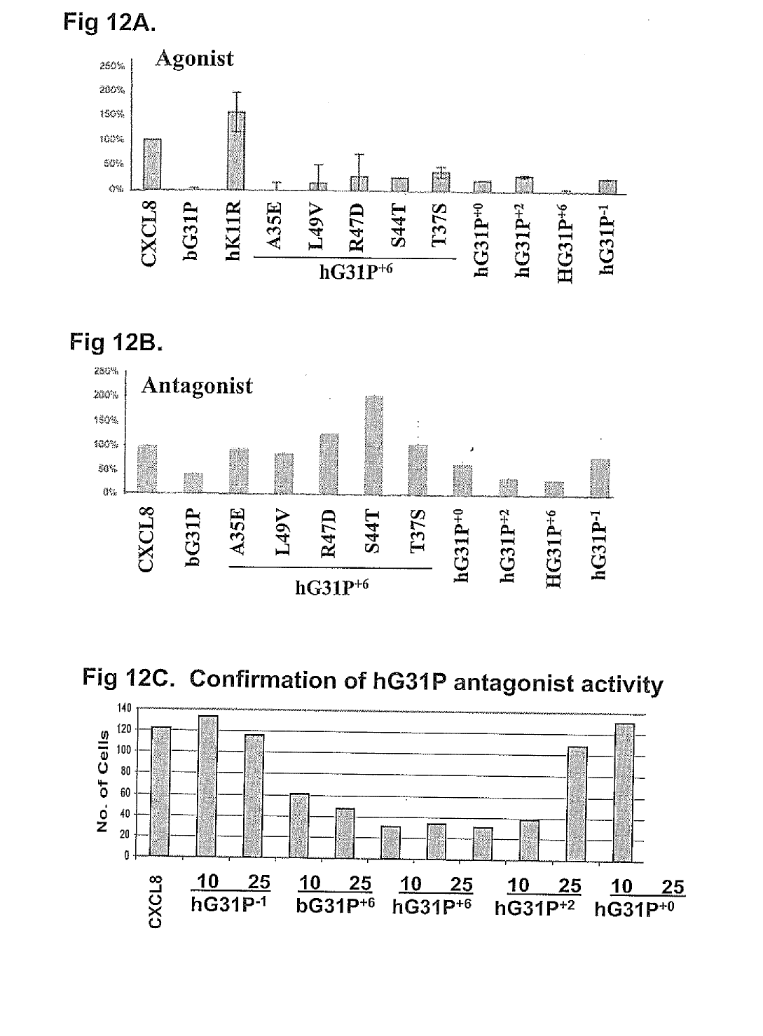

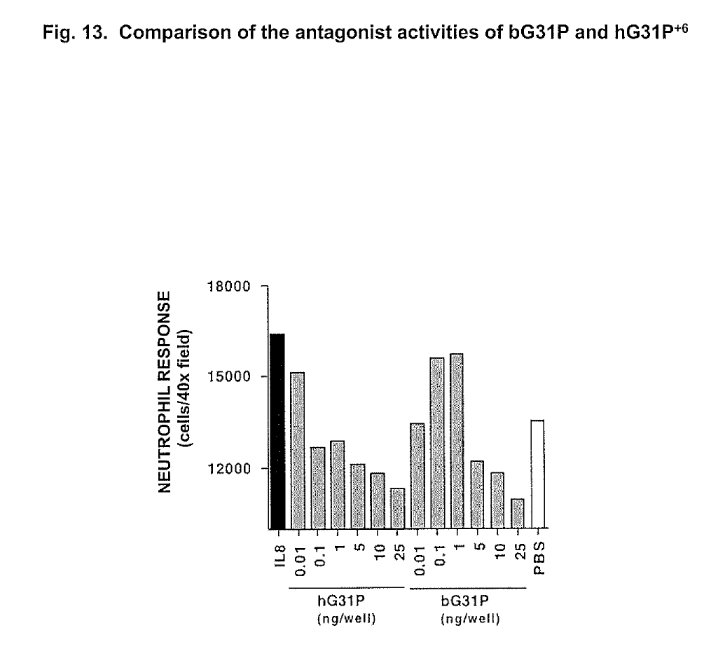

Biological characterization of the CXCL8 analogues. We used chemotaxis assays with purified human neutrophils to assess the agonist and CXCL8 antagonist activities of each construct (FIG. 12).

hCXCL8₍₃₋₇₂₎K11R (hK11R)-hK11R had significantly higher specific activity (neutrophil chemotaxis assays) than human CXCL8, such that it represents a much stronger neutrophil agonist than the native human chemokine. Thus, hK11R could be used in clinical situations calling for augmented neutrophil recruitment/activation.

hCXCL8₍₃₋₇₂₎K11R/G31P (hG31P)-G31P substitution within hK11R⁺⁶ essentially eliminated the agonist activity of this molecule, such that hG31P⁺⁶ was at least as effective an antagonist of CXCL8-driven neutrophil chemotaxis as bG31P⁺⁶.

The various N-terminal structures of hG31P did affect the biological activity of the analogues, such that hG31P⁺⁶ and hG31P⁺² appeared to be superior antagonists of CXCL8 chemotaxis, while hG31P⁺⁰ and hG31P⁺² possessed significantly less chemotaxis antagonist activity relative to bG31P⁺⁶. Interestingly, when compared for their abilities to inhibit CXCL8-induced ROI release from human neutrophils, the various N-terminal sequences had much less effect on the analogue's antagonist activities, with each analogue displaying highly significant ROI release antagonist activity. ROI release is dependent on the activity of NADPH oxidase in neutrophils, and it has been reported that NADPH oxidase is under the control of the CXCR2, but not the CXCR1.

The rationale for the different degrees of effectiveness of these various N-terminal substitution analogues may be that the extra two or six residues on hG31P⁺² or hG31P⁺⁶ may further reduce the potential for the ELR motif (i.e. on G31P) interaction with the CXCR1/CXCR2, perhaps by steric hinderance.

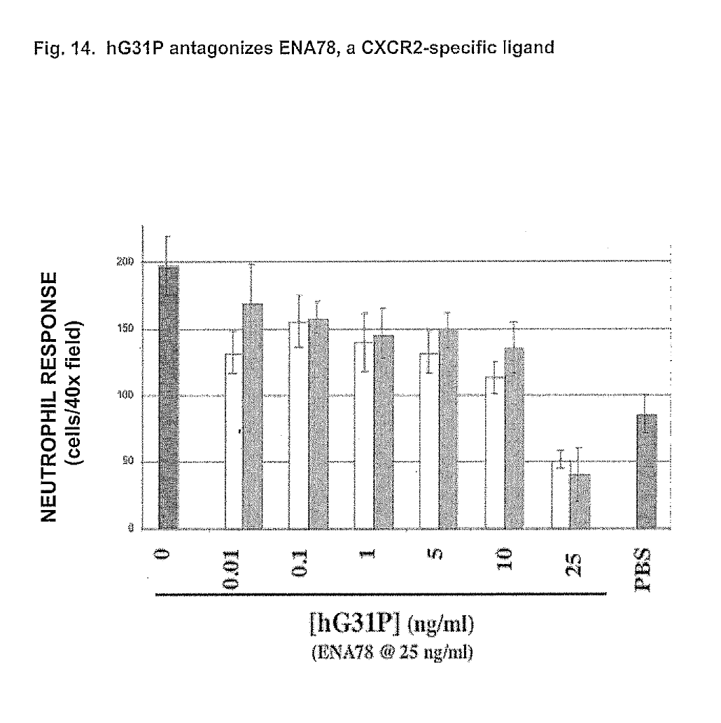

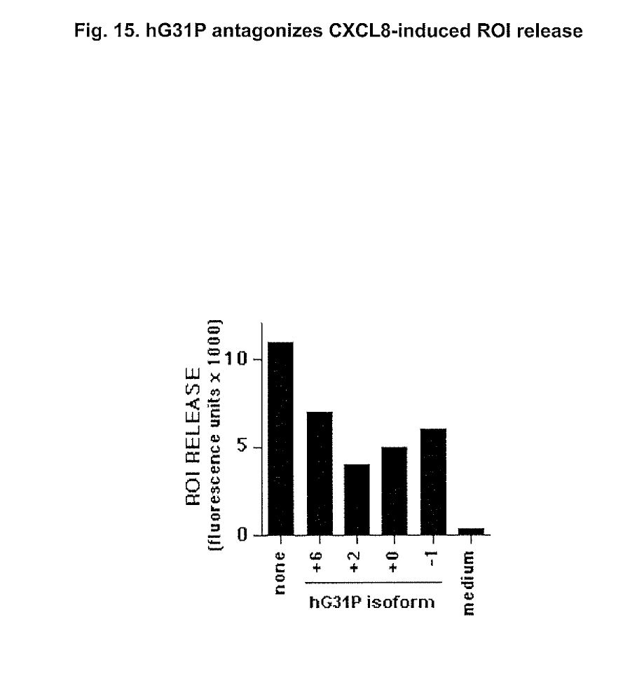

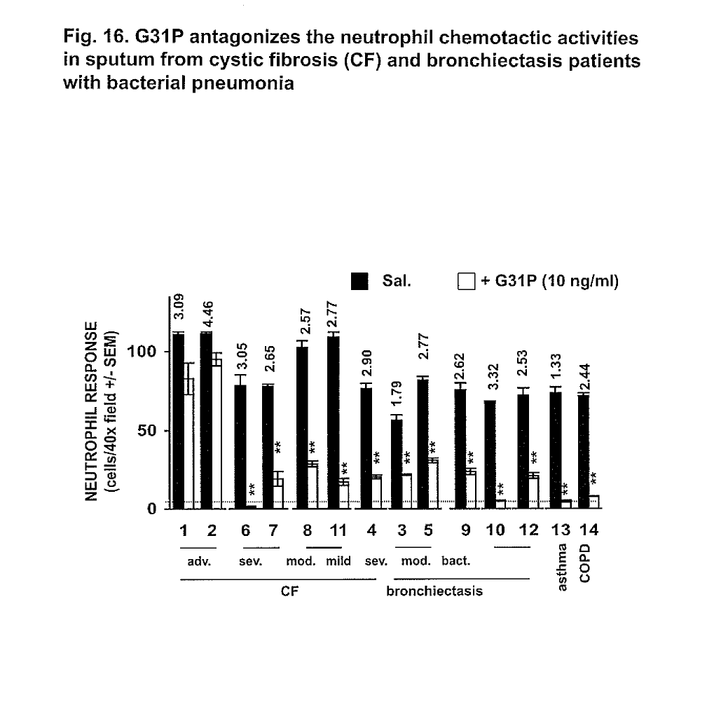

In order to further document the abilities of hG31P to antagonize CXCR2 functions, we assessed its abilities to inhibit CXCL5 (ENA-78)-dependent neutrophil chemotaxis; CXCL5 is a CXCR2-, but not CXCR1-, specific ligand. hG31P⁺⁶ antagonized its chemotactic activity in a dose-dependent fashion (FIG. 14).

We also assessed whether the various N-terminal analogues of hG31P possessed significant agonist activity, relative to PBS alone in a neutrophil chemotaxis assay. We found no significant responses on the part of the neutrophils to any of the alternate N-terminal analogues.

We next assessed whether additional substitutions within hG31P⁺⁶ of the human-bovine discrepant amino acids would augment this hG31P's antagonist activity. We generated and tested hG31P/A35E, hG31P/L49V, hG31P/R47D, hG31P/S44T and hG31P/T37S and found that all were ineffective agonists. Introduction of bovine-equivalent amino acids into positions 35, 37, 44, 47 or 49 differentially reduced the antagonist activity of these analogues, such that the R47D and S44T analogues seemed to augment the activity of CXCL8, rather than inhibiting it, while the A35E, L49V and T37S analogues displayed no significant chemotaxis inhibition activity.

We previously documented that bG31P could antagonize the neutrophil chemotactic activities present in sputum from cystic fibrosis (CF) patients undergoing bacterial exacerbations of pneumonia. Thus, we tested the ability of hG31P to block the neutrophil chemotactic activities present in sputum from patients with mild (n=1), moderate (n=2), severe (n=2), or advanced (n=2) CF, but also from patients with unclassified (n=2; bact), moderate (n=2), or severe (n=2) bronchiectasis. Sputa from patients diagnosed with asthma, COPD, or general sinusitis/bronchitis were also run with or without hG31P⁺⁶. With the exception of the one control asthma patient, all others were culture-positive for various bacterial species (e.g., Haemophilus, Pseudomonas, Staphylococcus). Each sample was titrated in preliminary experiments to determine the optimal dilution for use in the chemotaxis assay. G31P was effective to markedly effective in blocking the neutrophil chemotactic activities present in all samples, except those from the advanced CF patients. This suggests that an alternative etiology may exist for the pathology observed in advanced CF patients.

hG31P Anatgonizes Neutrophil Inflammation In Vivo

We previously documented the ability of bG31P to block endotoxin-induced dermal neutrophilic inflammation and airway endotoxemia pathology. Thus, we next assessed the activity of hG31P in models of airway endotoxemia.

Mouse Model of Airway Endotoxemia

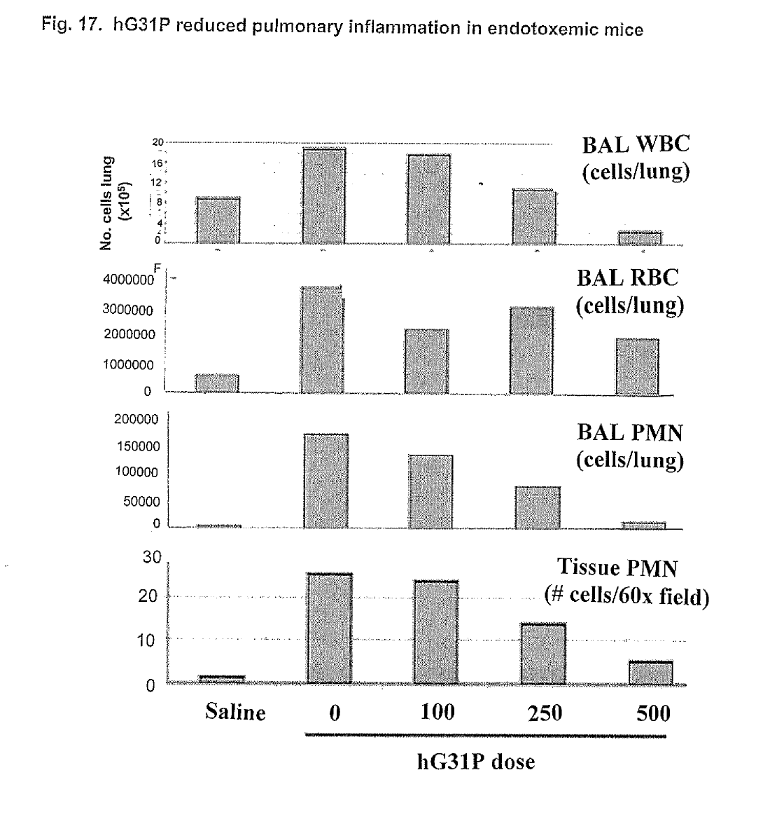

Inasmuch as mice are small animals and this would require substantially less G31P than guinea pigs (our standard model), we first tested the protective effects of hG31P⁺⁶ on neutrophilic inflammation in mice. In preliminary tests we determined that a LPS dose of 1.5 mg/kg provided an appropriate challenge dose for BALB/c mice; this is in stark contrast to guinea pigs, wherein 5 μg/kg of LPS induces a strong airway neutrophilia, pyrexia, and a pulmonary pleural hemorrhagic response. In a single mouse experiment (n=5/group), mice were given saline or 100, 250, or 500 μg/kg hG31P⁺⁶ s.c., then one hour later they were challenged intranasally, under light isofluorane gas anesthesia, with LPS (⅕ mg/kg). After 16 hours, the animals were euthanized using CO₂, then blood, bronchial alveolar lavage (BAL) fluid, and lung tissues were taken for analysis. While a previous pilot trial had shown that 150 μg/kg of bG31P had little effect in a LPS peritonitis model, hG31P⁺⁶ was highly effective in reducing airway total white blood cell and neutrophil infiltration, pulmonary parenchymal neutrophilia and, to a lesser and variable extent, also the appearance of red blood cells in the airways (FIG. 17).

Guinea Pig Model of Airway Endotoxemia

We also employed a guinea pig model of airway endotoxemia in three experiments. In one, the animals were given a supr-optimal dose of endotoxin (50 μg/kg), while in the second and third they were challenged with the standard LPS dose of 5 μg/kg, but with G31P delivered at varying levels. In each experiment, the animals were challenged intranasally with LPS and treated s.c. at the same time with G31P. Fifteen hours later the animals were sacrificed and their peripheral blood (WBC differentials) and pulmonary response (BAL WBC numbers, differentials, and neutrophil degranulation product levels, and tissue neutrophilia) were assessed, as above.

High (Morbid) Dose LPS Challenge Experiment

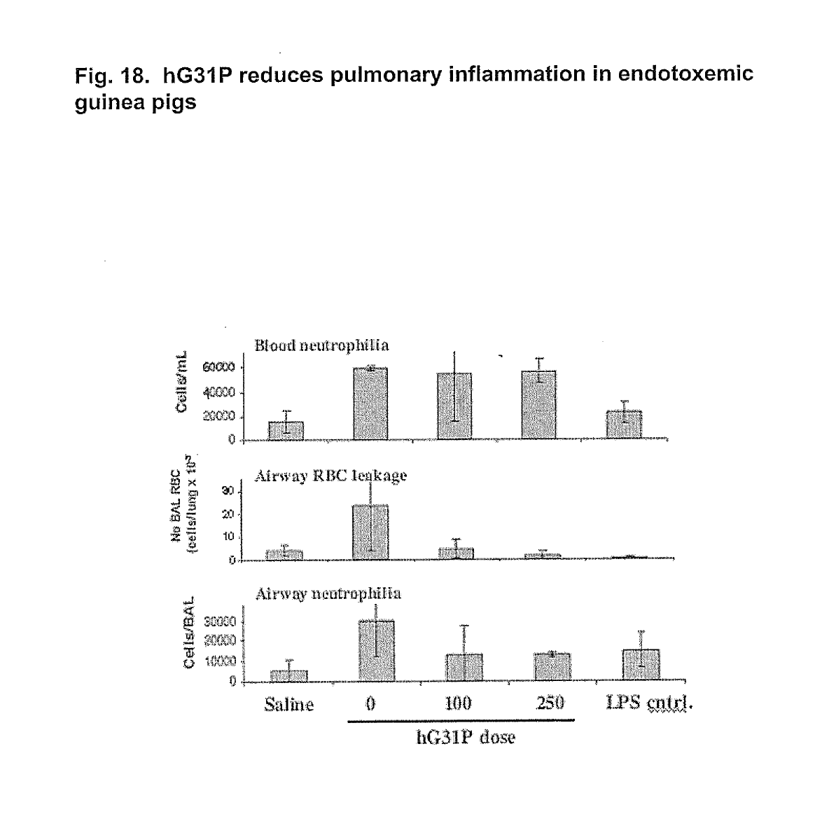

At doses of ≧50 μg/kg, LPS causes severe pulmonary damage in guinea pigs, including severe bleeding into the airways, although at these doses neutrophil infiltration of the lungs is blunted, relative to that observed with +/−5 μg/kg LPS. We found that even with 50 μg/kg LPS challenge, hG31P⁺⁶ was very effective in reducing the appearance of red blood cell (RBC) in the airway (FIG. 18). The G31P treatment also reduced the mean numbers of neutrophils in the BAL.

Efficacy of hG31P and hbG31P in Airway Endotoxemia Pathology

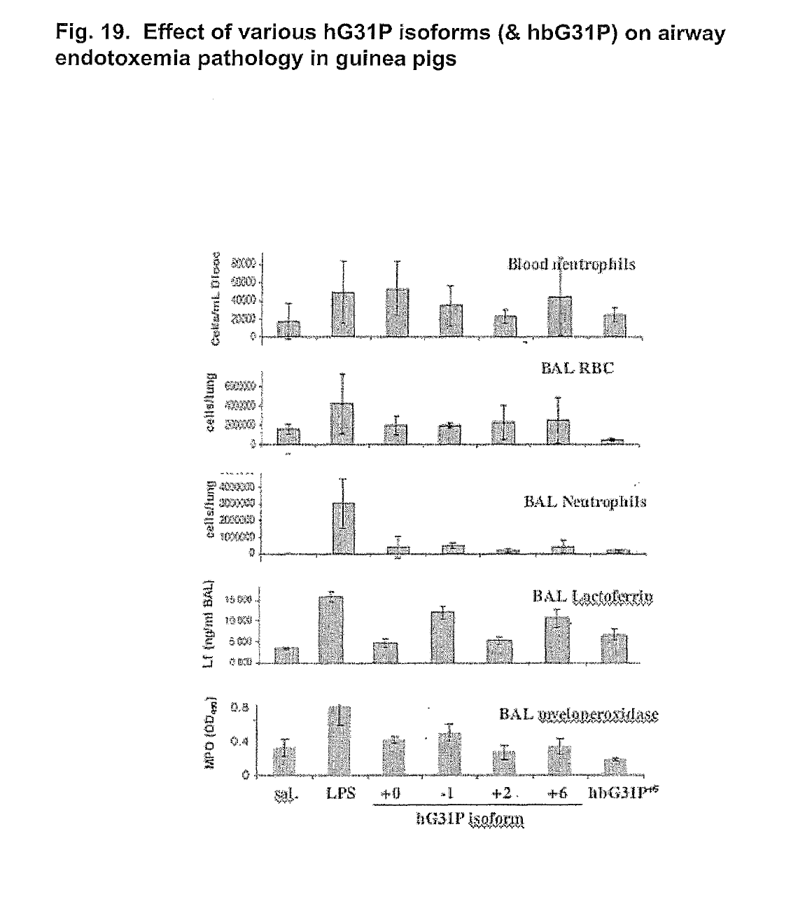

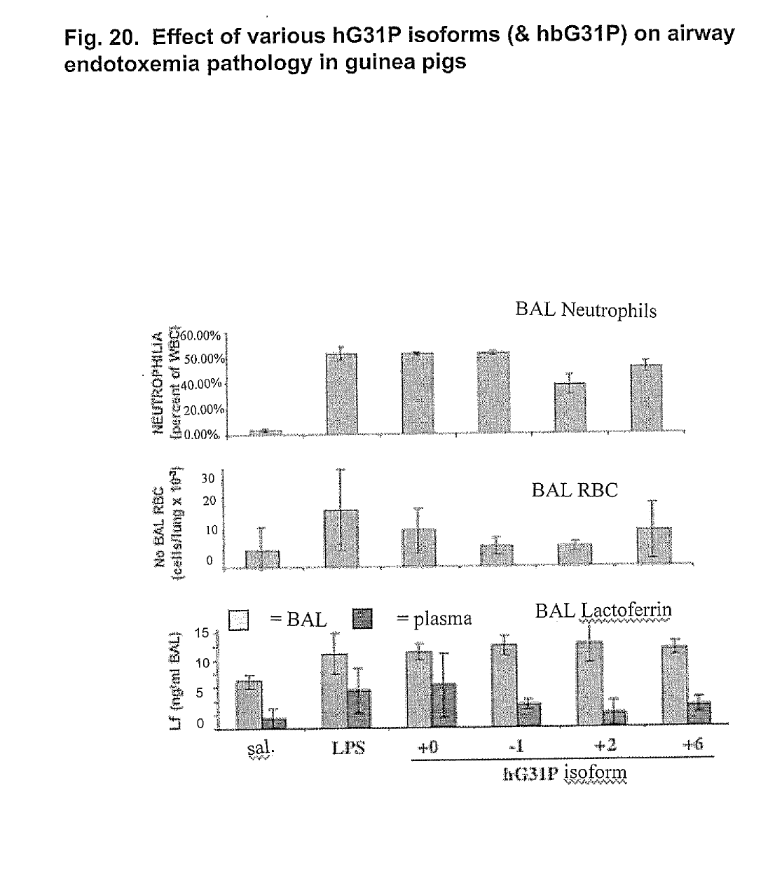

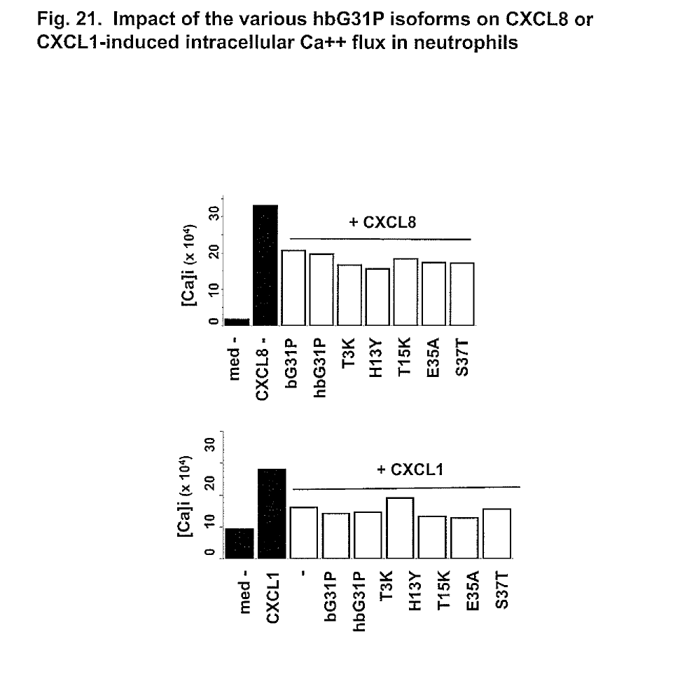

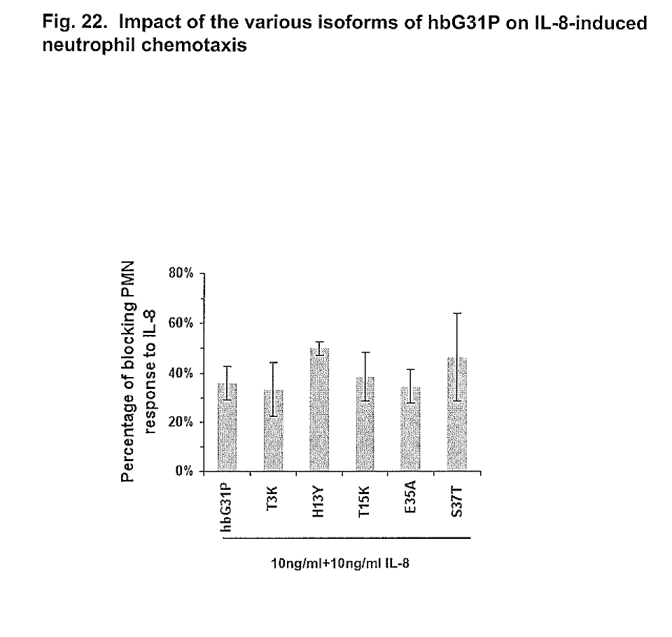

We compared the relative therapeutic efficacy of hG31P⁺⁰, hG31P⁻¹, hG31P⁺², and hG31P⁺⁶, as well as the bovine-human chaemera bhG31P⁺⁶, which had appeared to be highly effective in vitro against human CXCL8 dependent neutrophil recruitment. Again, we delivered 250 μg/kg of each G31P isoform to groups of guinea pigs (s.c.; n=5), and challenged them via airway with 5 μg/kg E. coli LPS, then 15 h later euthanized the animals and assessed their peripheral blood neutrophilia, and airway neutrophil and red blood cell levels, as well as the levels of two nuetrophil granule markers, lactoferrin (a neutrophil 1° granule-specific marker) and myeloperoxidase (a neutrophil 2° granule-specific marker). At this dose of 250 μg/kg, each of the G31P isoforms tested essentially ablated the infiltration of neutrophils into the airways (BAL neutrophils), and significantly decreased the appearance of RBC, lactoferrin and myeloperoxidase in the BAL (FIG. 19). It appeared as if hG31P⁺² may have given greater protection than the others, but not significantly so when all parameters are taken into account. The bhG31P chaemera was also highly effective in this system in terms of each of the parameters assessed.

### Toxicology Tests

Preliminary testing of bG31P toxicity has been performed. Delivery of bG31P (250 μg/kg) to three guinea pigs in a time-frame designed to optimize immune sensitization (i.e., if the molecule were an immunogen) did not cause any observable fluctuations from normal in the serum levels of a panel of liver and kidney enzymes. No changes in animal behavior or overall health were observed. Furthermore, preliminary histologic assessments of heart, kidney, lung, liver, gut, and from these animals revealed no evidence of inflammatory infiltrates, or local cell apoptosis or proliferation, or histopathologic abnormalities. We are making arrangements for an anatomic pathologist to independently assess these tissues. We were not able to detect discernible levels of anti-bG31P antibody reactivity in the serum of these animals, although the assays employed are not particularly sensitive.

Of importance to the generation of hG31P or bG31P using a prokaryotic (i.e., bacterial) expression system, we found that the preparations are significantly contaminated with bacterial endotoxin. When passed over commercial endotoxin-removal columns to reduce the endotoxin load of the drug, we found that the use of the endotoxin-removal columns resulted in unacceptably high loss of G31P. In vivo, treatment of guinea pigs with the levels of endotoxin found in therapeutic doses of G31P did not mimic the therapeutic activity of G31P.

Efficacy Studies in one experiment we employed hG31P⁺⁶ at 50 μg/kg (ie, 20% of the optimal dose for bG31P). Guinea pigs were challenged with 5 μg/kg of LPS and treated with this low dose of hG31P. We found that hG31P⁺² and hG31P⁺⁶ retained only modest efficacy in terms of reducing neutrophil infiltration, but was still effective in reducing red blood cell migration into the airways.

In another in vivo approach using the airway endotoxemia model, we challenged guinea pigs with a dose of LPS known to induce severe pulmonary hemorrhage, and treated them with 250 μg/kg hG31P. We knew from previous studies that at this elevated dose of LPS neutrophils are only poorly recruited from the vasculature into the airways.

In one experiment we employed hG31P⁺⁶ at 50 μg/kg (ie, 20% of the optimal dose for bG31P). Guinea pigs were challenged with 5 μg/kg of LPS and treated with this low dose of hG31P. We found that hG31P⁺² and hG31P⁺⁶ retained only modest efficacy in terms of reducing neutrophil infiltration, but was still effective in reducing red blood cell migration into the airways.

In another in vivo approach using the airway endotoxemia model, we challenged guinea pigs with 50 μg/kg of LPS, a dose known to induce severe pulmonary hemorrhage, and treated them with 250 μg/kg hG31P (see FIG. 18). We knew from previous studies that at this elevated dose of LPS neutrophils are only poorly recruited from the vasculature into the airways. Nevertheless, hG31P did have significant effects on reducing the high level of extravasation of RBC into the airway observed in saline-treated, high dose LPS-challenged animals (FIG. 18), and tended to reduce the airway neutrophil response and peripheral blood neutrophil mobilization associated with this challenge (FIG. 18).

We demonstrated herein that CXCL8₍₃₋₇₄₎K11R/G31P is a high affinity antagonist of multiple ELR-CXC chemokines. In vitro, this antagonist effectively blocked all of the neutrophil chemotactic activities expressed in mild to intense inflammatory lesions within two mucosal compartments (lungs, mammary glands), and up to 97% blocked endotoxin-induced inflammatory responses in vivo. We identified CXCL8 as a major chemoattractant in the pneumonia and mastitis samples, but also demonstrated that 35% of the activity in the bacterial pneumonia samples was due to non-CXCL8 chemoattractants that were also effectively antagonized by CXCL8₍₃₋₇₄₎K11R/G31P. Based on studies of inflammatory responses in rodents (Tateda et al., 2001; Tsai et al., 2000), cattle (Caswell et al., 1997), and humans (Villard et al., 1995), it is clear that these samples could contain numerous ELR⁺ CXC chemokines (e.g., CXCL5, and CXCL8) to which CXCL8₍₃₋₇₄₎K11R/G31P has an antagonistic effect.

### Example I

### pJ5:G03799 Plasmid Construction

Referring to FIG. 1, this plasmid includes the following features:

(SEQ ID No. 40)

transcription terminator
ACTAGTCTAGCATAACCCCTTGGGGCCTCTAAACGGGTCTTGAGGGGTTT
TTTG

(SEQ ID No. 41)

Trc promoter lacO
GGTAGCTTGACAATTAATCATCGGCTCGTATAATGTGTGGAATTGTGAGC
GGATAACAATTCCACACAGGAGGATAACATATGGGCTCTAAAGAACTGCG
TTGTCAATGCATTCGTACTTACTCTAAGCCATTCCACCCGAAGTTCATCA
AAGAACTGCGTGTGATTGAATCTCCGCCACACTGCGCCAATACCGAAATC
ATTGTTAAACTGAGCGACGGTCGTGAACTGTGTCTGGACCCGAAAGAAAA
TTGGGTACAGCGTGTGGTGGAAAAATTTCTGAAACGTGCCGAAAACTCTT
GATAATCTAGAGAATTC

(SEQ ID No. 42)

pT-T7
CTAGCATAACCCCTTGGGGCCTCTAAACGGGTCTTGAGGGGTTTTTTG

(SEQ ID No. 43)

pP-trc
TTGACAATTAATCATCGGCTCGTATAAT

(SEQ ID No. 44)

lacO
GTGTGGAATTGTGAGCGGATAACAATTCC

(SEQ ID No. 45)

pRBS-SD + 7Nde
ACACAGGAGGATAACAT
Start
ATG

(SEQ ID No. 2)

hK11RG31P + 2
GGCTCTAAAGAACTGCGTTGTCAATGCATTCGTACTTACTCTAAGCCATT
CCACCCGAAGTTCATCAAAGAACTGCGTGTGATTGAATCTCCGCCACACT
GCGCCAATACCGAAATCATTGTTAAACTGAGCGACGGTCGTGAACTGTGT
CTGGACCCGAAAGAAAATTGGGTACAGCGTGTGGTGGAAAAATTTCTGAA
ACGTGCCGAAAACTCT
Stop
TGA
Stop
TAA

### General Notes:

Transcriptional promoter pTrc ends at −7 position from mRNA start site
lacO adds 4 nt to 5′ end so −1/+1 of mRNA is GA. Also adds C on the 3′ end to complete palindrome
RBS has ACAC on 5′ end to complete palindrome for lad binding
Notes for pRBS-SD+7Nde
Consensus ribosome binding site plus 7 base spacer that places an NdeI site at the initiation AUG

### Translation Map

### Start

| 1 | ATG |
|---|---|
| 1 | M |
| hK11RC31P + 2 | |
| 1 | GGCTCTAAAGAACTGCGTTGTCAATGCATTCGTACTTACTCTAAGCCATTCCACCCGAAG |
| 1 | G S K E L R C Q C I R T Y S K P F H P K |
| 61 | TTCATCAAAGAACTGCGTGTGATTGAATCTCCGCCACACTGCGCCAATACCGAAATCATT |
| 21 | F I K E L R V I E S P P H C A N T E I I |
| 121 | GTTAAACTGAGCGACGGTCGTGAACTGTGTCTGGACCCGAAAGAAAATTGGGTACAGCGT |
| 41 | V K L S D G R E L C L D P K E N W V Q R |
| 181 | GTGGTGGAAAAATTTCTGAAACGTGCCGAAAACTCT |
| 61 | V V E K F L K R A E N S |
| Stop | |
| 1 | TGA |
| 1 | * |
| Stop | |
| 1 | TAA |
| 1 | * |

Nucleotide sequence is SEQ ID No. 2
Amino acid sequence is SEQ ID No. 1

### Example II

### E. coli BL21/G31P-pJ5 Vector Tranformation Procedure

1. Add 100 μL ddH₂O to 2 μg plasmid (pJ5:G03799) (plasmid solution)
2. Grow the stock cell culture BL21 in 100 mL LB at 37° C. until O.D. ˜0.6
3. Centrifuge the cultures in a 50 mL sterile tube at 2000 rpm 4° C. (7 min) and discard supernatant
4. Add 15 mL TFB1 buffer to resuspend the pellet for 90 min in ice
5. Centrifuge at 2000 rpm 4° C. for 5 min and discard supernatant
6. Add 4 mL TFB2 to resuspend the pellet
7. Divide the competent cell suspension into 1.5 mL eppendorf tubes, each containing 250 μL
8. Add 10 μL plasmid solution (from 1) to each eppendorf tube in ice for 90 min
9. Heat shock at 42° C. for 80 seconds and then immediately place in ice.
10. Transfer the cells to 15 mL sterile tube with 7 mL LB, and then incubate at 37° C. 200 rpm for 1.5 hours
11. Spread 200 μL culture on 90 mm agar plate containing 100 μg/mL kanamycin Incubate the plate at 37° C. for 12-16 hours
12. Transfer about 10 colonies from the plate to 1.5 mL eppendorf with 1 mL LB and kanamycin
13. Incubate overnight at 37° C.
14. Place 200 μL of each 1 mL culture into eppendorf tube containing 800 μL LB and kanamycin for test (remaining cultures were stored at 4° C.)
15. Incubate at 37° C. for 3 hours and then induced with 2-5 μL IPTG(400 mM) for 4 hours
16. Centrifuge the test cultures and one culture without pJ5 vector control at 12000 rpm for 1 min
17. Discard the supernatant and add 8M Urea for 1 hour to lyse the bacterial cells
18. Compare the lysate by SDS-PAGE analysis for G31P formation
19. Check which cultures samples are transformed by pJ5 vector

### Note

TFB1:100 mM RbCl, 50 mM Mn 30 mM KAc, 10 mM CaCl₂, 15% glycerol, pH 5.8
TFB2:100 mM MOPS, 10 mM RbCl, 75 mM CaCl₂, 15% glycerol, pH 8.0
Kanamycin stock: 0.1 mg kanamycin sodium/ml
Kanamycin concentration: 1/1000 kanamycin stock(v/v)

### Example III

### Research Scale Manufacturing Protocol for Human IL8/K11R/G31P:BL21/pJ5 system

### Cell Growth:

1. Using a 500 ml Erlenmeyer flask and a sterile loop, inoculate 100 ml LB/w Kan (100 micrograms/ml) with 1 ml cell culture stored at −70° C. Grow o/n at 37° C. with shaking over night.
2. Inoculate each 500 ml LB/w Kan (in a 2 L Erlenmeyer flask) with 5 ml of the overnight culture. Grow at 37° C. in a shaker incubator until OD600=0.8 (should be about 3 hours).
3. Add inducing agent (IPTG) to a final concentration of 0.2 mM. Continue to grow in the shaker incubator at 37° C. for 5 hours. Using 100 microliters of culture, test the OD once every hour to determine growth curve.
4. Remove 1 ml of culture for SDS-PAGE analysis.
5. Centrifuge at 4° C., 6K rpm, 15 minutes.
6. Cell pellet can be frozen or continue directly to sonication step (cell lysis).

### Cell Lysis Process:

1. Resuspend in sonication buffer (50 mM Tris, pH 8.0, 1 mM EDTA, 700 mM NaCl, 1 mM PMSF), each liter of cell culture pellet was resuspended in 100 mL sonication buffer. Add lysozyme to final concentration of 200 micrograms/ml and TritonX-100 to a final concentration of 0.5%.
2. Leave at room temperature for 1 hour.
3. Sonicate or French press to lyse cells. Sonication is performed on ice over a total time of 7 minutes using 15 second pulses (15 seconds on, 15 seconds off) at level 3.5.
4. Heat the sonicated solution in an 80° C. water bath for 10 minutes.
5. Cool immediately on ice for 15-30 minutes.
6. Centrifuge at 4° C., 14K rpm, 45 minutes.
7. Collect supernatant and label sample as S1.
8. Dialyze S1 to 20 mM citrate buffer pH=6.0 at 4° C. over 8 hours. (Label resulting sample as S1:citrate)
9. Resuspend the pellet in 8 M urea for loading on SDS-PAGE to indicate the level of G31P left.

### Purification Process:

### SP Sepharose Fast Flow, Pharmacia Biotech

### Code No. 17-0729-01

Note: 1. The glass column is about 28 mm diameter.

2. 10 mL of SP Resin is packed in each column
3. Each SP column could be used for 200 mL S1 (pellet lysant of 2 liters cell culture)
1. Pre-equilibrate the SP column with 20 mM citrate buffer, pH 6.0.
2. Load S1 citrate onto the SP column slowly over 2-3 hours.
3. Wash with 300 ml 20 mM Citrate, pH 6.0
4. Wash with 100 ml 20 mM Citrate, pH 6.0, 60 mM NaCl
5. Wash with 100 ml 20 mM Citrate, pH 6.0, 120 mM NaCl
6. Wash with 50 ml 20 mM Citrate, pH 6.0, 180 mM NaCl
7. Wash with 80 ml 20 mM Citrate, pH 6.0, 180 mM NaCl, 1% w/v Triton X114

Note: to reduce endotoxin level from ˜15000 EU/mg G31P to less than 100 EU/mg

8. Wash with 200 ml 20 mM Citrate, pH 6.0, 180 mM NaCl
9. Wash with 50 ml 20 mM Citrate, pH 6.0, 240 mM NaCl
10. Elute with 150 ml 20 mM Citrate, pH 6.0, 600 mM NaCl
Note: Whether Wash or Elute procedure, add 10 mL buffer each time and total to the final volume
11. Run on a 15% SDS-PAGE gel under reducing conditions to identify which fractions contain G31P.
12. Pool positive fractions (almost are the eluted buffer).
13. Concentrate and change to appropriate storage buffer using a 50 ml Amicon with a YM1 membrane.

### General Production Level Obtained:

For BL21 host and pJ5 vector this procedure produces approximately 12 mg G31P per liter cell culture.

### Example III

### E. coli BL21(DE3)Gold/G31P-pET22b Vector Transformation Procedure

1. Add 100 μL ddH2O to 2 μg plasmid (G31P-pET22b) (plasmid solution)
2. Grow the stock cell culture BL21(DE3)Gold in 100 mL LB at 37° C. until O.D. ˜0.6
3. Centrifuge the cultures in a 50 mL sterile tube at 200 rpm 4° C. (7 min) and discard supernatant
4. Add 15 mL TFB1 buffer to resuspend the pellet for 90 min in ice
5. Centrifuge at 2000 rpm 4° C. for 5 min and discard supernatant
6. Add 4 mL TFB2 to resuspend the pellet
7. Divide the competent cell suspension into 1.5 mL eppendorf tubes, each containing 250 μL
8. Add 10 μL plasmid solution (from 1) to each eppendorf tube in ice for 90 min
9. Heat shock at 42° C. for 80 seconds and then immediately place in ice.
10. Transfer the cells to 15 mL sterile tube with 7 mL LB, and then incubate at 37° C. 200 rpm for 1.5 hours
11. Spread 200 mL culture on 90 mm agar plate containing 100 μg/mL ampicillin (AP)
12. Incubate the plate at 37° C. for 12-16 hours
13. Transfer about 10 colonies from the plate to 1.5 mL eppendorf with 1 mL LB and ampicillin
14. Incubate overnight at 37° C.
15. Place 200 μL of each 1 mL culture into eppendorf tube containing 800 μL LB and ampicillin for test (remaining cultures were stored at 4° C.)
16. Incubate at 37° C. for 3 hours and then induced with 2-5 μL IPTG (400 mM) for 4 hours
17. Centrifuge the test cultures and one culture without G31P-pET22b vector at 12000 rpm for 1 min
18. Discard the supernatant and add 8M Urea for 1 hour to lyse the bacterial cells
19. Compare the lysate by SDS-PAGE analysis for G31P formation
20. Check which cultures samples are transformed by G31P-pET22b vector

### Note

TFB1: 100 mM RbCl, 50 mM Mn 30 mM KAc, 10 mM CaCl₂, 15% glycerol, pH 5.8
TFB2: 100 mM MOPS, 10 mM RbCl, 75 mM CaCl₂, 15% glycerol, pH 8.0
Ampicillin stock: 0.1 mg ampicillin sodium/ml
Ampicillin concentration: 1/1000 ampicillin stock(v/v)

### Example IV

### Research Scale Manufacturing Protocol for human IL8/K11R/G31P:BL21 (DE3)Gold/pET22b System

### Cell Growth:

1. Using a 500 ml Erlenmeyer flask and a sterile loop, inoculate 100 ml LB/w AP (100 micrograms/ml) with 1 ml cell culture stocked at −70° C. Grow o/n at 37° C. with shaking over night.
2. Inoculate each 500 ml LB/w AP (in a 2 L Erlenmeyer flask) with 5 ml of the overnight culture. Grow at 37° C. in a shaker incubator until OD600=0.8 (should be about 3 hours).
3. Add inducing agent (IPTG) to a final concentration of 0.2 mM. Continue to grow in the shaker incubator at 37° C. for 5 hours. Using 100 microliters of culture, test the OD once every hour to determine growth curve.
4. Remove 1 ml of culture for SDS-PAGE analysis.
5. Centrifuge at 4° C., 6K rpm, 15 minutes.
6. Cell pellet can be frozen or continue directly to sonication step (cell lysis).

### Cell Lysis Process:

1. Resuspend in sonication buffer (50 mM Tris, pH 8.0, 1 mM EDTA, 700 mM NaCl, 1 mM PMSF), each liter of cell culture pellet was resuspended by 100 mL sonication buffer. Add lysozyme to final concentration of 200 micrograms/ml and TritonX-100 to a final concentration of 0.5%.
2. Leave at room temperature for 1 hour.
3. Sonicate or French press to lyse cells. Sonication is performed on ice over a total time of 7 minutes using 15 second pulses (15 seconds on, 15 seconds off) at level 3.5.
4. Heat the sonicated solution in an 80° C. water bath for 10 minutes.
5. Cool immediately on ice for 15-30 minutes.
6. Centrifuge at 4° C., 14K rpm, 45 minutes.
7. Collect supernatant and label sample as S1.
8. Dialyze S1 to 20 mM citrate buffer pH=6.0 at 4° C. over 8 hours. (Label resulting sample as S1:citrate)
9. Resuspend the pellet in 8 M urea for loading on SDS-PAGE to indicate the level of G31P left.

### Purification Process:

### SP Sepharose Fast Flow, Pharmacia Biotech

### Code No. 17-0729-01

Note: 1. The glass column is about 28 mm diameter.
2. 10 mL of SP Resin is packed in each column
3. Each SP column could be used for 200 mL S1 (pellet lysant of 2 liters cell culture)
1. Pre-equilibrate the SP column with 20 mM citrate buffer, pH 6.0.
2. Load S1:citrate onto the SP column slowly over 2-3 hours.
3. Wash with ˜300 ml 20 mM Citrate, pH 6.0
4. Wash with 100 ml 20 mM Citrate, pH 6.0, 60 mM NaCl
5. Wash with 100 ml 20 mM Citrate, pH 6.0, 120 mM NaCl

6. Wash with 50 ml 20 mM Citrate, pH 6.0, 180 mM NaCl

7. Wash with 80 ml 20 mM Citrate, pH 6.0, 180 mM NaCl, 1% w/v Triton X114
Note: to reduce endotoxicin level from ˜15000 EU/mg G31P to less than 100 EU/mg
8. Wash with 200 ml 20 mM Citrate, pH 6.0, 180 mM NaCl
9. Wash with 50 ml 20 mM Citrate, pH 6.0, 240 mM NaCl
10. Elute with 150 ml 20 mM Citrate, pH 6.0, 600 mM NaCl
Note: Whether Wash or Elute procedure, add 10 mL buffer each time and total to the final volume
11. Run on a 15% SDS-PAGE gel under reducing conditions to identify which fractions contain G31P.
12. Pool positive fractions (almost are the eluted buffer).
13. Concentrate and change to appropriate storage buffer using a 50 ml Amicon with a YM1 membrane.

### General Production Level Obtained:

For BL21(DE3)Gold host and pET22b vector, this procedure produces approximately 15 mg G31P per liter cell culture.

While the preferred embodiments of the invention have been described above, it will be recognized and understood that various modifications may be made therein, and the appended claims are intended to cover all such modifications which may fall within the spirit and scope of the invention.

### REFERENCES

1. Baggiolini, M. 1998. Chemokines and leukocyte traffic. Nature. 392:565-568.
2. Sekido, N., N. Mukaida, A. Harada, I. Nakanishi, Y. Watanabe, and K. Matsushima. 1993. Prevention of lung reperfusion injury in rabbits by a monoclonal antibody against interleukin-8. Nature. 365:654-657.
3. Villard, J., F. Dayer Pastore, J. Hamacher, J. D. Aubert, S. Schlegel Haueter, and L. P. Nicod. 1995. GRO alpha and interleukin-8 in Pneumocystis carinii or bacterial pneumonia and adult respiratory distress syndrome. Am. J. Respir. Crit. Care Med. 152:1549-1554.
4. Mukaida, N., T. Matsumoto, K. Yokoi, A. Harada, and K. Matsushima. 1998. Inhibition of neutrophil-mediated acute inflammation injury by an antibody against interleukin-8 (IL-8). Inflamm. Res. 47 (supple 3):S151-157.
5. Harada, A., N. Mukaida, and K. Matsushima. 1996. Interleukin 8 as a novel target for intervention therapy in acute inflammatory diseases. Inflamm. Res. 2:482-489.
6. Walley, K. R., N. W. Lukacs, T. J. Standiford, R. M. Strieter, and S. L. Kunkel. 1997. Elevated levels of macrophage inflammatory protein 2 in severe murine peritonitis increase neutrophil recruitment and mortality. Infect. Immun. 65:3847-3851.
7. Slocombe, R., J. Malark, R. Ingersoll, F. Derksen, and N. Robinson. 1985. Importance of neutrophils in the pathogenesis of acute pneumonic pasteurellosis in calves. Am. J. Vet. Res, 46:2253.
8. Caswell, J. L., D. M. Middleton, S. D. Sorden, and J. R. Gordon. 1997. Expression of the neutrophil chemoattractant interleukin-8 in the lesions of bovine pneumonic pasteurellosis. Vet. Pathol. 35:124-131.
9. Caswell, J. L., D. M. Middleton, and J. R. Gordon. 2001. The importance of interleukin-8 as a neutrophil chemoattractant in the lungs of cattle with pneumonic pasteurellosis. Canad. J. Vet. Res. 65:229-232.
10. Baggiolini, M., and B. Moser. 1997. Blocking chemokine receptors. J. Exp. Med. 186:1189-1191.
11. Ahuja, S. K., and P. M. Murphy, 1996. The CXC chemokines growth-regulated oncogene (GRO) alpha, GRObeta, GROgamma, neutrophil-activating peptide-2, and epithelial cell derived neutrophil-activating peptide-78 are potent agonists for the type B, but not the type A, human interleukin-8 receptor. J. Biol. Chem. 271:20545-20550.
12. Loetscher, P., M. Seitz, I. Clark Lewis, M. Baggiolini, and B. Moser. 1994. Both interleukin-8 receptors independently mediate chemotaxis. Jurkat cells transfected with IL-8R1 or IL-8R2 migrate in response to IL-8, GRO alpha and NAP-2. FEBS Lett. 341:187-192.
13. Wuyts, A., P. Proost, J. P. Lenaerts, A. Ben Baruch, J. Van Damme, and J. M. Wang. 1998. Differential usage of the CXC chemokine receptors 1 and 2 by interleukin-8, granulocyte chemotactic protein-2 and epithelial-cell-derived neutrophil attractant-78. Eur. J. Biochem. 255:67-73.
14. Richardson, R., B. Pridgen, B. Haribabu, H. Ali, and R. Snyderman. 1998. Differential cross-regulation of the human chemokine receptors CXCR1 and CXCR2. Evidence for time-dependent signal generation. J. Biol. Chem. 273:23830-23836.
15. McColl, S. R., and I. Clark Lewis. 1999. Inhibition of murine neutrophil recruitment in vivo by CXC chemokine receptor antagonists. J. Immunol. 163:2829-2835.
16. Jones, S. A., M. Wolf, S. Qin, C. R. Mackay, and M. Baggiolini. 1996. Different functions for the interleukin 8 receptors (IL-8R) of human neutrophil leukocytes: NADPH oxidase and phospholipase D are activated through IL-8R1 but not IL-8R2. Proc. Natl. Acad. Sci. U.S.A. 93:6682-6686.
17. White, J. R., J. M. Lee, P. R. Young, R. P. Hertzberg, A. J. Jurewicz, M. A. Chaikin, K. Widdowson, J. J. Foley, L. D. Martin, D. E. Griswold, and H. M. Sarau. 1998. Identification of a potent, selective non-peptide CXCR2 antagonist that inhibits interleukin-8-induced neutrophil migration. J. Biol. Chem. 273:10095-10098.
18. Tateda, K., T. A. Moore, M. W. Newstead, W. C. Tsai, X. Zeng, J. C. Deng, G. Chen, R. Reddy, K. Yamaguchi, and T. J. Standiford. 2001. Chemokine-dependent neutrophil recruitment in a murine model of Legionella pneumonia: potential role of neutrophils as immunoregulatory cells. Infect. Immun. 69:2017-2024.
19. Tsai, W. C., R. M. Strieter, B. Mehrad, M. W. Newstead, X. Zeng, and T. J. Standiford. 2000. CXC chemokine receptor CXCR2 is essential for protective innate host response in murine Pseudomonas aeruginosa pneumonia. Infect. Immun. 68:4289-4296.
20. Goodman, R. B., R. M. Strieter, C. W. Frevert, C. J. Cummings, P. Tekamp Olson, S. L. Kunkel, A. Walz, and T. R. Martin. 1998. Quantitative comparison of C-X-C chemokines produced by endotoxin-stimulated human alveolar macrophages. Am. J. Physiol. 275:L87-95.
21. Nufer, O., M. Corbett, and A. Walz. 1999. Amino-terminal processing of chemokine ENA-78 regulates biological activity. Biochem. 38:636-642.
22. Wuyts, A., A. D'Haese, V. Cremers, P. Menten, J. P. Lenaerts, A. De Loof, H. Heremans, P. Proost, and J. Van Damme. 1999. NH2- and COOH-terminal truncations of murine granulocyte chemotactic protein-2 augment the in vitro and in vivo neutrophil chemotactic potency. J. Leukoc. Biol. 163:6155-6163.
23. Clark Lewis, I., B. Dewald, M. Loetscher, B. Moser, and M. Baggiolini. 1994. Structural requirements for interleukin-8 function identified by design of analogs and CXC chemokine hybrids. J. Biol. Chem. 269:16075-16081.
24. Li, F., and J. R. Gordon. 2001. IL-8.sub.(3-74)K11R is a high affinity agonist of the neutrophil CXCR1 and CXCR2. Biochem. Biophys. Res. Comm. 286:595-600.
25. Moser, B., B. Dewald, L. Barella, C. Schumacher, M. Baggiolini, and I. Clark Lewis. 1993. Interleukin-8 antagonists generated by N-terminal modification. J. Biol. Chem. 268:7125-7128.
26. Caswell, J. L., D. M. Middleton, and J. R. Gordon. 1998. Production and functional characterization of recombinant bovine interleukin-8 as a neutrophil-activator and specific chemoattractant in vitro and in vivo, Vet. Immunol. Immunopath. 67:327-340.
27. Coligan, J., A. Kruisbeek, D. Margulies, E. Shevach, and W. Strober. 1994. Current Protocols in Immunology. John Wiley & Sons, New York.
28. Waller, K. P. 1997. Modulation of endotoxin-induced inflammation in the bovine teat using antagonists/inhibitors to leukotrienes, platelet activating factor and interleukin 1 beta. Vet. Immunol. Immunopathol. 57:239-251.
29. Cairns, C. M., J. R. Gordon, F. Li, M. E. Baca-Estrada, T. N. Moyana, and J. Xiang. 2001. Lymphotactin expression by engineered myeloma tumor cells drives tumor regression. Mediation by CD4+ and CD8+ T cells and neutrophils expressing XCR1 receptors. J. Immunol. 167:57-65.
30. Gordon, J. R. 2000. TGFb and TNFa secretion by mast cells stimulated via the FceRI activates fibroblasts for high level production of monocyte chemoattractant protein-1. Cell Immunol. 201:42-49.
31. Gordon, J. R., and S. J. Galli. 1994. Promotion of mouse fibroblast collagen gene expression by mast cells stimulated via the FceRI. Role for mast cell-derived transforming growth factor-b and tumor necrosis factor-a. J. Exp. Med. 180:2027-2037.
32. Gordon, J. R. 2000. Monocyte chemoattractant protein-1 (MCP-1) expression during cutaneous allergic responses in mice is mast cell-dependent and largely mediates monocyte recruitment. J. Allergy Clin. Immunol. 106:110-116.
33. Caswell, J. L. 1998. The role of interleukin-8 as a neutrophil chemoattractant in bovine bronchopneumonia. Ph.D. thesis, Department of Veterinary Pathology, University of Saskatchewan. 241 pg.
34. Hochreiter, W. W., R. B. Nadler, A. E. Koch, P. L. Campbell, M. Ludwig, W. Weidner, and A. J. Schaeffer, 2000. Evaluation of the cytokines interleukin-8 and epithelial neutrophil activating peptide-78 as indicators of inflammation in prostatic secretions. Urology. 56:1025-1029.
35. Persson, K. I. Larsson, and C. Hallen Sandgren. 1993. Effects of certain inflammatory mediators on bovine neutrophil migration in vivo and in vitro. Vet. Immunol. Immunopathol. 37:99-112.
36. Gray, G. D., K. A. Knight, R. D. Nelson, and M. Herron, J. 1982. Chemotactic requirements of bovine leukocytes. Am. J. Vet. Res. 43:757-759.
37. Fernandez, H. N., P. M. Henson, A. Otani, and T. E. Hugli. 1978. Chemotactic response to human C3a and C5a anaphylatoxins. I. Evaluation of C3a and C5a leukotaxis in vitro and under stimulated in vivo conditions. J. Immunol. 120:109-115.
38. Riollet, C., P. Rainard, and B. Poutrel. 2000. Differential induction of complement fragment C5a and inflammatory cytokines during intramammary infections with Escherichia coli and Staphylococcus aureus. Clin. Diagn. Lab Immunol. 7:161-167.
39. Shuster, D. E., M. E. Kehrli, Jr., P. Rainard, and M. Paape. 1997. Complement fragment C5a and inflammatory cytokines in neutrophil recruitment during intramammary infection with Escherichia coli. Infect. Immun. 65:3286-3292.
40. Bless, N. M., R. L. Warner, V. A. Padgaonkar, A. B. Lentsch, B. J. Czermak, H. Schmal, H. P. Friedl, and P. A. Ward. 1999. Roles for C-X-C chemokines and C5a in lung injury after hindlimb ischemia-reperfusion. Am. J. Physiol. 276:L57-63.
41. Ember, J. A., S. D. Sanderson, T. E. Hugli, and E. L. Morgan. 1994. Induction of interleukin-8 synthesis from monocytes by human C5a anaphylatoxin. Am. J. Pathol. 144:393-403.
42. Fisher, C., G. Slotman, S. Opal, J. Pribble, R. Bone, G. Emmanuel, D. Ng, D. Bloedow, and M. Catalano. 1994. Initial evaluation of human recombinant interleukin-1 receptor antagonist in the treatment of sepsis syndrome: a randomized, open-label, placebocontrolled multicenter trial. The IL-1RA Sepsis Syndrome Study Group. Crit. Care Med. 22:11-21.
43. Verbon, A., P. E. Dekkers, T. ten Hove, C. E. Hack, J. Pribble, T. Turner, S. Souza, T. Axtelle, F. Hoek, S.-J. van Deventer, and T. van der Poll. 2001. IC14, an anti-CD 14 antibody, inhibits endotoxin-mediated symptoms and inflammatory responses in humans. J. Immunol. 166:3599-3605.
44. Clark Lewis, I., K. S. Kim, K. Rajarathnam, J. H. Gong, B. Dewald, B. Moser, M. Baggiolini, and B. D. Sykes. 1995. Structure-activity relationships of chemokines. J. Leukoc. Biol. 57:703-711.
45. Jones, S. A., B. Dewald, I. Clark Lewis, and M. Baggiolini. 1997. Chemokine antagonists that discriminate between interleukin-8 receptors. Selective blockers of CXCR2. J. Biol. Chem. 272:16166-16169.
46. Hang, L., B. Frendeus, G. Godaly, and C. Svanborg. 2000. Interleukin-8 receptor knockout mice have subepithelial neutrophil entrapment and renal scarring following acute pyelonephritis. J. Infect. Dis. 182:1738-1748.
47. Saurer, L., P. Reber, T. Schaffner, M. W. Buchler, C. Buri, A. Kappeler, A. Walz, H. Friess, and C. Mueller. 2000. Differential expression of chemokines in normal pancreas and in chronic pancreatitis. Gastroenterol. 118:356-367.
48. Szekanecz, Z., R. M. Strieter, S. L. Kunkel, and A. E. Koch. 1998. Chemokines in rheumatoid arthritis. Springer Semin. Immunopathol. 20:115-132.
49. MacDermott, R. P. 1999. Chemokines in the inflammatory bowel diseases. J. Clin. Immunol. 19:266-272.
50. Damas, J. K., L. Gullestad, T. Ueland, N. O, Solum, S, Simonsen, S. S. Froland, and P. Aukrust. 2000. CXC-chemokines, a new group of cytokines in congestive heart failure—possible role of platelets and monocytes. Cardiovasc. Res. 45:428-436.
51. Morsey, M., Y. Popowych, J. Kowalski, G. Gerlach, D. Godson, M. Campos, and L. Babiuk. 1996. Molecular cloning and expression of bovine interleukin-8. Microbial Pathogen. 20:203-212.
52. Benson, M., I. L. Strannegard, G. Wennergren, and O, Strannegard. 1999. Interleukin-5 and interleukin-8 in relation to eosinophils and neutrophils in nasal fluids from school children with seasonal allergic rhinitis. Pediatr. Allergy Immunol. 10:178-185.
53. Hauser, U., M. Wagenmann, C. Rudack, and C. Bachert. 1997. Specific immunotherapy suppresses IL-8-levels in nasal secretions: A possible explanation for the inhibition of eosinophil migration. Allergol. 20:184-191.
54. Sehmi, R., O. Cromwell, A. J. Wardlaw, R. Moqbel, and A. B. Kay. 1993. Interleukin-8 is a chemoattractant for eosinophils purified from subjects with a blood eosinophilia but not from normal healthy subjects. Clin. Exp. Allergy 23:1027-1036.
55. Ulfinan, L. H., D. P. Joosten, J. A. van der Linden, J. W. Lammers, J. J. Zwaging a, and L. Koenderman. 2001. IL-8 induces a transient arrest of rolling eosinophils on human endothelial cells. J. Immunol. 166:588-595.

1. An expression vector comprising a polynucleotide sequence deduced from an amino acid sequence as set forth in SEQ ID No. 1 operatively linked to a suitable promoter.

2. The expression vector according to claim 1 wherein the polynucleotide sequence is a polynucleotide sequence as set forth in SEQ ID No. 2.

3. A method of producing hG31P⁺² peptide comprising:
transforming a suitable host cell with an expression vector comprising a polynucleotide sequence deduced from an amino acid sequence as set forth in SEQ ID No. 1 operatively linked to a suitable promoter functional in said host;
growing the host cell under conditions promoting expression of the hG31P⁺²; and
recovering the hG31P⁺² from the host cell.

4. The method according to claim 3 wherein the polynucleotide sequence is a polynucleotide sequence as set forth in SEQ ID No. 2.

5. A method of treating a CXC chemokine-mediated disease comprising administering to an individual in need of such treatment an effective amount of a peptide produced according to the method of claim 3.

6. The method according to claim 5 wherein the chemokine mediated disease is selected from the group consisting of psoriasis, atopic dermatitis, osteo arthritis, rheumatoid arthritis, asthma, chronic obstructive pulmonary disease, adult respiratory distress syndrome, inflammatory bowel disease, Crohn's disease, ulcerative colitis, stroke, septic shock, multiple sclerosis, endotoxic shock, gram negative sepsis, toxic shock syndrome, cardiac and renal reperfusion injury, glomerulonephritis, thrombosis, graft vs. host reaction, Alzheimer's disease, allograft rejections, malaria, restenosis, angiogenesis, atherosclerosis, osteoporosis, gingivitis and undesired hematopoietic stem cells release and diseases caused by respiratory viruses, herpes viruses, and hepatitis viruses, meningitis, cystic fibrosis, pre-term labor, cough, pruritus, multi-organ dysfunction, trauma, strains, sprains, contusions, psoriatic arthritis, herpes, encephalitis, CNS vasculitis, traumatic brain injury, CNS tumors, subarachnoid hemorrhage, post surgical trauma, interstitial pneumonitis, hypersensitivity, crystal induced arthritis, acute and chronic pancreatitis, acute alcoholic hepatitis, necrotizing enterocolitis, chronic sinusitis, uveitis, polymyositis, vasculitis, acne, gastric and duodenal ulcers, celiac disease, esophagitis, glossitis, airflow obstruction, airway hyperresponsiveness, bronchiolitis obliterans organizing pneumonia, bronchiectasis, bronchiolitis, bronchiolitis obliterans, chronic bronchitis, cor pulmonae, dyspnea, emphysema, hypercapnea, hyperinflation, hypoxemia, hyperoxia-induced inflammations, hypoxia, surgical lung volume reduction, pulmonary fibrosis, pulmonary hypertension, right ventricular hypertropy, sarcoidosis, small airway disease, ventilation-perfusion mismatching, wheeze, colds and lupus.


SEQUENCE LISTING (45 sequences)

SEQ ID NO: 1 - protein, 72 aa, artificial
  synthetic human ELR CXC peptide
GSKELRCQCIRTYSKPFHPKFIKELRVIESPPHCANTEIIVKLSDGRELCLDPKENWVQRVVEKFLKRAENS

SEQ ID NO: 2 - DNA, 216 bp, artificial
  DNA sequence of artificial human G31P+2 construct
ggctctaaagaactgcgttgtcaatgcattcgtacttactctaagccattccacccgaagttcatcaaagaactgcgtgtgattgaatctccgccacactgcgccaataccgaaatcattgttaaactgagcgacggtcgtgaactgtgtctggacccgaaagaaaattgggtacagcgtgtggtggaaaaatttctgaaacgtgccgaaaactct

SEQ ID NO: 3 - DNA, 2432 bp, artificial
  plasmid sequence
gaggaagcggaaggcgagagtagggaactgccaggcatcaaactaagcagaaggcccctgacggatggcctttttgcgtttctacaaactctttctgtgttgtaaaacgacggccagtcttaagctcgggcctcaaataatgattttagattaacggtctccttttgaattctctagattatcaagagttttcggcacgtttcagaaatttttccaccacacgctgtacccaattttctttcgggtccagacacagttcacgaccgtcgctcagtttaacaatgatttcggtattggcgcagtgtggcggagattcaatcacacgcagttctttgatgaacttcgggtggaatggcttagagtaagtacgaatgcattgacaacgcagttctttagagcccatatgttatcctcctgtgtggaattgttatccgctcacaattccacacattatacgagccgatgattaattgtcaagctagccaaaaaacccctcaagacccgtttagaggccccaaggggttatgctagactagtccccagagaccgttaatcgccatccagctgatattccctatagtgcatggtcatagctgtttcctggcagctctggcccgtgtctcaaaatctctgatgttacattgtacaagataaaataatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagc

SEQ ID NO: 4 - protein, 72 aa, artificial
  Bovine 3-74 K11RG31P synthetic peptide
TELRCQCIRTHSTPFHPKFIKELRVIESPPHCENSEIIVKLTNGNEVCLNPKEKWVQKVVQVFVKRAEKQDP

SEQ ID NO: 5 - protein, 72 aa, artificial
  bovine 3-74 K11R P32G synthetic peptide
TELRCQCIRTHSTPFHPKFIKELRVIESGGHCENSEIIVKLTNGNEVCLNPKEKWVQKVVQVFVKRAEKQDP

SEQ ID NO: 6 - protein, 72 aa, artificial
  bovine - 3-74 T12SH13PG31P synthetic peptide
TELRCQCIRSPSTPFHPKFIKELRVIESPPHCENSEIIVKLTNGNEVCLNPKEKWVQKVVQVFVKRAEKQDP

SEQ ID NO: 7 - protein, 72 aa, artificial
  bovine - 3-74 T12SH13PG31PP32G synthetic peptide
TELRCQCIRSPSTPFHPKFIKELRVIESPGHCENSEIIVKLTNGNEVCLNPKEKWVQKVVQVFVKRAEKQDP

SEQ ID NO: 8 - protein, 72 aa, artificial
  bhG31P synthetic peptide
GSTELRCQCIRTHSTPFHPKFIKELRVIESPPHCENSEIIVKLTDGRELCLDPKENWVQRVVEKFLKRAENS

SEQ ID NO: 9 - protein, 70 aa, artificial
  hG31P (0) synthetic peptide
KELRCQCIRTYSKPFHPKFIKELRVIESPPHCANTEIIVKLSDGRELCLDPKENWVQRVVEKFLKRAENS

SEQ ID NO: 10 - protein, 69 aa, artificial
  hG31P (-1)  synthetic peptide
ELRCQCIRTYSKPFHPKFIKELRVIESPPHCANTEIIVKLSDGRELCLDPKENWVQRVVEKFLKRAENS

SEQ ID NO: 11 - protein, 72 aa, artificial; identical to SEQ ID NO: 1
  hG31P (+2) synthetic peptide

SEQ ID NO: 12 - protein, 76 aa, artificial
  hG31P (+6) synthetic peptide
GSMGGSKELRCQCIRTYSKPFHPKFIKELRVIESPPHCANTEIIVKLSDGRELCLDPKENWVQRVVEKFLKRAENS

SEQ ID NO: 13 - protein, 72 aa, artificial
  hG31P K3T synthetic peptide
GSTELRCQCIRTYSKPFHPKFIKELRVIESPPHCANTEIIVKLSDGRELCLDPKENWVQRVVEKFLKRAENS

SEQ ID NO: 14 - protein, 72 aa, artificial
  hG31P Y13H synthetic peptide
GSKELRCQCIRTHSKPFHPKFIKELRVIESPPHCANTEIIVKLSDGRELCLDPKENWVQRVVEKFLKRAENS

SEQ ID NO: 15 - protein, 72 aa, artificial
  hG31P K15T (0) synthetic peptide
GSKELRCQCIRTYSTPFHPKFIKELRVIESPPHCANTEIIVKLSDGRELCLDPKENWVQRVVEKFLKRAENS

SEQ ID NO: 16 - protein, 72 aa, artificial
  hG31P A35E(0) synthetic peptide
GSKELRCQCIRTYSKPFHPKFIKELRVIESPPHCENTEIIVKLSDGRELCLDPKENWVQRVVEKFLKRAENS

SEQ ID NO: 17 - protein, 72 aa, artificial
  hG31P T37S (0) synthetic peptide
GSKELRCQCIRTYSKPFHPKFIKELRVIESPPHCANSEIIVKLSDGRELCLDPKENWVQRVVEKFLKRAENS

SEQ ID NO: 18 - protein, 72 aa, artificial
  hG31P S44T (0) synthetic peptide
GSKELRCQCIRTYSKPFHPKFIKELRVIESPPHCANTEIIVKLTDGRELCLDPKENWVQRVVEKFLKRAENS

SEQ ID NO: 19 - protein, 72 aa, artificial
  hG31P R47D (0) synthetic peptide
GSKELRCQCIRTYSKPFHPKFIKELRVIESPPHCANTEIIVKLSDGDELCLDPKENWVQRVVEKFLKRAENS

SEQ ID NO: 20 - protein, 72 aa, artificial
  hG31P N56K(0) synthetic peptide
GSKELRCQCIRTYSKPFHPKFIKELRVIESPPHCANTEIIVKLSDGRELCLDPKEKWVQRVVEKFLKRAENS

SEQ ID NO: 21 - protein, 72 aa, artificial
  hG31P R60K synthetic peptide
GSKELRCQCIRTYSKPFHPKFIKELRVIESPPHCANTEIIVKLSDGRELCLDPKENWVQKVVEKFLKRAENS

SEQ ID NO: 22 - protein, 72 aa, artificial
  hG31P K64V (0) synthetic peptide
GSKELRCQCIRTYSKPFHPKFIKELRVIESPPHCANTEIIVKLSDGRELCLDPKENWVQRVVEVFLKRAENS

SEQ ID NO: 23 - protein, 72 aa, artificial
  hG31P L49V synthetic peptide
GSKELRCQCIRTYSKPFHPKFIKELRVIESPPHCANTEIIVKLSDGREVCLDPKENWVQRVVEKFLKRAENS

SEQ ID NO: 24 - protein, 72 aa, artificial
  bhG31P T3K synthetic peptide
GSKELRCQCIRTHSTPFHPKFIKELRVIESPPHCENSEIIVKLTDGRELCLDPKENWVQRVVEKFLKRAENS

SEQ ID NO: 25 - protein, 72 aa, artificial
  bhG31P H13Y synthetic peptide
GSTELRCQCIRTYSTPFHPKFIKELRVIESPPHCENSEIIVKLTDGRELCLDPKENWVQRVVEKFLKRAENS

SEQ ID NO: 26 - protein, 72 aa, artificial
  bhG31P T15K synthetic peptide
GSTELRCQCIRTHSKPFHPKFIKELRVIESPPHCENSEIIVKLTDGRELCLDPKENWVQRVVEKFLKRAENS

SEQ ID NO: 27 - protein, 72 aa, artificial
  bhG31P E35A synthetic peptide
GSTELRCQCIRTHSTPFHPKFIKELRVIESPPHCANSEIIVKLTDGRELCLDPKENWVQRVVEKFLKRAENS

SEQ ID NO: 28 - protein, 72 aa, artificial
  bhG31P S37T synthetic peptide
GSTELRCQCIRTHSTPFHPKFIKELRVIESPPHCENTEIIVKLTDGRELCLDPKENWVQRVVEKFLKRAENS

SEQ ID NO: 29 - protein, 76 aa, artificial
  bhG31P (+6) synthetic peptide
GSMGGSTELRCQCIRTHSTPFHPKFIKELRVIESPPHCENSEIIVKLTDGRELCLDPKENWVQRVVEKFLKRAENS

SEQ ID NO: 30 - protein, 72 aa, artificial; identical to SEQ ID NO: 8
  bhG31P (+2) synthetic peptide

SEQ ID NO: 31 - protein, 70 aa, artificial
  bhG31P (+0) synthetic peptide
TELRCQCIRTHSTPFHPKFIKELRVIESPPHCENSEIIVKLTDGRELCLDPKENWVQRVVEKFLKRAENS

SEQ ID NO: 32 - protein, 69 aa, artificial
  bhG31P (-1) synthetic peptide
ELRCQCIRTHSTPFHPKFIKELRVIESPPHCENSEIIVKLTDGRELCLDPKENWVQRVVEKFLKRAENS

SEQ ID NO: 33 - protein, 76 aa, artificial
  hK11R (+6) synthetic peptide
GSMGGSKELRCQCIRTYSKPFHPKFIKELRVIESGPHCANTEIIVKLSDGRELCLDPKENWVQRVVEKFLKRAENS

SEQ ID NO: 34 - protein, 76 aa, artificial
  hK11RG31P A35E(+6) synthetic peptide
GSMGGSKELRCQCIRTYSKPFHPKFIKELRVIESPPHCENTEIIVKLSDGRELCLDPKENWVQRVVEKFLKRAENS

SEQ ID NO: 35 - protein, 76 aa, artificial
  hK11RG31P T37S (+6) synthetic peptide
GSMGGSKELRCQCIRTYSKPFHPKFIKELRVIESPPHCANSEIIVKLSDGRELCLDPKENWVQRVVEKFLKRAENS

SEQ ID NO: 36 - protein, 76 aa, artificial
  hK11RG31P S44T (+6) synthetic peptide
GSMGGSKELRCQCIRTYSKPFHPKFIKELRVIESPPHCANTEIIVKLTDGRELCLDPKENWVQRVVEKFLKRAENS

SEQ ID NO: 37 - protein, 76 aa, artificial
  hK11RG31P R47D (+6) synthetic peptide
GSMGGSKELRCQCIRTYSKPFHPKFIKELRVIESPPHCANTEIIVKLSDGDELCLDPKENWVQRVVEKFLKRAENS

SEQ ID NO: 38 - protein, 76 aa, artificial
  hK11RG31P L49V synthetic peptide
GSMGGSKELRCQCIRTYSKPFHPKFIKELRVIESPPHCANTEIIVKLSDGREVCLDPKENWVQRVVEKFLKRAENS

SEQ ID NO: 39 - protein, 74 aa, artificial
  Bovine 3-74 K11RG31P (+2) synthetic peptide
GSTELRCQCIRTHSTPFHPKFIKELRVIESPPHCENSEIIVKLTNGNEVCLNPKEKWVQKVVQVFVKRAEKQDP

SEQ ID NO: 40 - DNA, 54 bp, artificial
  synthetic transcription termination sequence
actagtctagcataaccccttggggcctctaaacgggtcttgaggggttttttg

SEQ ID NO: 41 - DNA, 317 bp, artificial
  trc promoter and lac O artificial sequence
gctagcttgacaattaatcatcggctcgtataatgtgtggaattgtgagcggataacaattccacacaggaggataacatatgggctctaaagaactgcgttgtcaatgcattcgtacttactctaagccattccacccgaagttcatcaaagaactgcgtgtgattgaatctccgccacactgcgccaataccgaaatcattgttaaactgagcgacggtcgtgaactgtgtctggacccgaaagaaaattgggtacagcgtgtggtggaaaaatttctgaaacgtgccgaaaactcttgataatctagagaattc

SEQ ID NO: 42 - DNA, 48 bp, artificial
  pT-T7 promoter sequence
ctagcataaccccttggggcctctaaacgggtcttgaggggttttttg

SEQ ID NO: 43 - DNA, 28 bp, artificial
  trc promoter sequence
ttgacaattaatcatcggctcgtataat

SEQ ID NO: 44 - DNA, 29 bp, artificial
  lac O sequence
gtgtggaattgtgagcggataacaattcc

SEQ ID NO: 45 - DNA, 17 bp, artificial
  pRBS-SD+7Nde sequence
acacaggaggataacat